(19) United States
(12) Patent Application Publication
(10) Pub. No.: US 2026/0251662 A9
(43) Pub. Date: Aug. 27, 2026

(54) METHODS FOR MONITORING MOLECULAR BIOMARKERS FOR AGING AND DISEASE

(71) Applicant: The Board of Trustees of the Leland Stanford Junior University, Stanford, CA (US)

(72) Inventors: Vinit MAHAJAN, Los Altos, CA (US); Julian WOLF, Redwood City, CA (US)

(21) Appl. No.: 18/997,478

(22) PCT Filed: Sep. 25, 2023
(86) PCT No.: PCT/US2023/075029

§ 371 Date: Jan. 21, 2025

Related U.S. Application Data
(60) Provisional application No. 63/377,093, filed on Sep. 26, 2022.

(51) Int. Cl. G01N 33/68; G01N 33/5751

(52) U.S. Cl. CPC G01N 33/6893; G01N 33/5751; G01N 2800/16; G01N 2800/164; G01N 2800/50; G01N 2800/52

(57) ABSTRACT

Compositions, methods, and kits are provided for diagnosing vitreoretinal diseases and age-related pathologies. In particular, aqueous humor biomarkers have been identified that correlate with biological aging and age-related pathologies and morbidity. The use of such biomarkers may allow earlier intervention in treatment of aging-related diseases. In addition, methods of using aqueous humor biomarkers for prognosis, diagnosis, and monitoring treatment of vitreoretinal diseases are also provided.

### CROSS REFERENCE TO RELATED APPLICATION

This application claims benefit of U.S. Provisional Patent Application No. 63/377,093, filed Sep. 26, 2022, which application is incorporated herein by reference in its entirety.

### BACKGROUND OF THE INVENTION

Liquid biopsies of highly-accessible liquid tissues, such as blood and cerebrospinal fluid, not only reflect their physiology, but also surrounding healthy and diseased systems (Velez et al., Mol Cancer 20, 39 (2021); Ignatiadis et al., Nat Rev Clin Oncol 18, 297-312, (2021)). The aqueous humor (AH) in the anterior chamber of the eye has critical functions such as providing nutrition to anterior segment cells like the cornea and maintaining intraocular pressure (Goel et al., Open Ophthalmol J 4, 52-59, (2010)). However, its molecular physiology is still poorly understood, since the small sample volume, low protein concentration and sparse cellular components have so far limited its exploration. Analogous to blood and cerebrospinal fluid, one promising way to study the molecular physiology of complex fluids such as AH would be to explore their molecular composition using large-scale molecular tools such as proteomic profiling.

There remains a need for better methods of monitoring molecular pathophysiology and human health.

### SUMMARY OF THE INVENTION

Compositions, methods, and kits are provided for diagnosing vitreoretinal diseases and age-related pathologies. In particular, aqueous humor biomarkers have been identified that correlate with biological aging and age-related pathologies and morbidity. The use of such biomarkers may allow earlier intervention in treatment of aging-related diseases. In addition, the inventors have discovered that protein exchange occurs between the vitreous and aqueous humor of the eye, which enables monitoring of biomarkers of various vitreoretinal diseases by measuring levels of biomarkers in the aqueous humor.

In one aspect, a method of predicting biological age and determining risk of age-related pathology and morbidity in a patient is provided, the method comprising: obtaining an aqueous humor sample from an eye of the patient; measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein decreased levels of the one or more biomarkers selected from MMP10, NRP1, SEMA3C, HES5, and FGFRL1 compared to reference value ranges for the levels of the one or more biomarkers in a control aqueous humor sample indicate that the patient has a risk of age-related pathology and morbidity; and increasing screening of the patient for an aging-related disease if the patient is identified as having the risk age-related pathology and morbidity.

In certain embodiments, the method further comprises administering a treatment for the aging-related disease to the patient if the patient is identified as having the aging-related disease.

In certain embodiments, a method of monitoring biological aging of an eye in a patient is provided, the method comprising: obtaining an aqueous humor sample from an eye of the patient; and measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein levels of the one or more biomarkers selected from MMP10, NRP1, SEMA3C, HES5, and FGFRL1 are correlated with biological age of the eye.

In certain embodiments, the levels of at least two, at least three, or at least four biomarkers selected from MMP10, NRP1, SEMA3C, HES5, and FGFRL1 are measured in the aqueous humor sample. In some embodiments, the levels of MMP10, NRP1, SEMA3C, HES5, and FGFRL1 are measured in the aqueous humor sample.

In certain embodiments, the measuring the levels of the biomarkers comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot. In some embodiments, the levels of the biomarkers are measured using a multiplex aptamer array.

In another aspect, a kit comprising agents for detecting at least 3 biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) is provided. In some embodiments, the kit comprises agents for detecting all of the MMP10, NRP1, SEMA3C, HES5, and FGFRL1 biomarkers is provided.

In certain embodiments, the kit further comprises reagents for performing an aptamer-based proteomic assay or immunoassay. In certain embodiments, the kit comprises an aptamer or antibody that specifically binds to MMP10, an aptamer or antibody that specifically binds to NRP1, an aptamer or antibody that specifically binds to SEMA3C, an aptamer or antibody that specifically binds to HES5, and an aptamer or antibody that specifically binds to FGFRL1.

In certain embodiments, the further comprises instructions for predicting biological age and determining risk of age-related pathology and morbidity in a patient or determining the biological age of the eye.

In another aspect, a protein selected from the group consisting of stromelysin-2 (MMP10), neuropilin-1 (NRP1), semaphorin 3C (SEMA3C), hes family bHLH transcription factor 5 (HES5), and fibroblast growth factor receptor like 1 (FGFRL1) for use as a biomarker in predicting biological age and determining risk of age-related pathology and morbidity is provided.

In another aspect, a protein selected from the group consisting of stromelysin-2 (MMP10), neuropilin-1 (NRP1), semaphorin 3C (SEMA3C), hes family bHLH transcription factor 5 (HES5), and fibroblast growth factor receptor like 1 (FGFRL1) for use as a biomarker for determining the biological age of an eye is provided.

In another aspect, an in vitro method of predicting biological age and determining risk of age-related pathology and morbidity is provided, the method comprising: obtaining an aqueous humor sample from an eye of the patient; and measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein decreased levels of the one or more biomarkers selected from MMP10, NRP1, SEMA3C, HES5, and FGFRL1 compared to reference value ranges for the levels of the one or more biomarkers in a control aqueous humor sample indicate that the patient has a risk of age-related pathology and morbidity.

In another aspect, a method of diagnosing and treating age-related macular degeneration (AMD) in a patient is provided, the method comprising: obtaining an aqueous humor sample from an eye of the patient; measuring levels of one or more biomarkers selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) in the aqueous humor sample, wherein increased levels of the one or more biomarkers selected from SERPINF1, VEGFA, FLT1, VEGFB, and VEGFD compared to reference value ranges for the levels of the one or more biomarkers in a control aqueous humor sample indicate that the patient has AMD; and treating the patient for the AMD if the patient has a positive diagnosis for AMD.

In certain embodiments, the levels of at least two, at least three, or at least four biomarkers selected from SERPINF1, VEGFA, FLT1, VEGFB, and VEGFD are measured in the aqueous humor sample. In some embodiments, the levels of SERPINF1, VEGFA, FLT1, VEGFB, and VEGFD are measured in the aqueous humor sample.

In certain embodiments, treatment of the patient for the AMD comprises administering vitamin C, zinc, vitamin E, copper, beta-carotene, lutein, zeaxanthin, ranibizumab, aflibercept, brolucizumab, or faricimab, or a combination thereof to the patient.

In certain embodiments, measuring the levels of the biomarkers comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot. In some embodiments, the levels of the biomarkers are measured using a multiplex aptamer array.

In certain embodiments, the subject has not yet developed clinical symptoms. In other embodiments, the subject has developed clinical symptoms.

In another aspect, a method of monitoring AMD in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) in the aqueous humor sample, wherein detection of increased levels of the one or more biomarkers selected from SERPINF1, VEGFA, FLT1, VEGFB, and VEGFD in the second aqueous humor sample compared to the first aqueous humor sample indicate that the patient is worsening, and wherein detection of decreased levels of the one or more biomarkers selected from SERPINF1, VEGFA, FLT1, VEGFB, and VEGFD in the second aqueous humor sample compared to the first aqueous humor sample indicate that the patient is improving.

In another aspect, a method of monitoring efficacy of a treatment of a patient for AMD is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD); and evaluating the efficacy of the treatment, wherein detection of increased levels of the one or more biomarkers selected from SERPINF1, VEGFA, FLT1, VEGFB, and VEGFD in the second aqueous humor sample compared to the first aqueous humor sample indicate that the patient is worsening or not responding to the treatment, and detection of decreased levels of the one or more biomarkers selected from SERPINF1, VEGFA, FLT1, VEGFB, and VEGFD in the second aqueous humor sample compared to the first aqueous humor sample indicate that the patient is improving.

In certain embodiments, the method further comprises altering the treatment if the patient is worsening or not responding to the treatment.

In another aspect, a kit for diagnosing AMD is provided, the kit comprising agents for detecting at least 3 biomarkers selected from the group consisting of serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD). In certain embodiments, the kit comprises agents for detecting all of the SERPINF1, VEGFA, FLT1, VEGFB, and VEGFD biomarkers.

In certain embodiments, the kit further comprises reagents for performing an aptamer-based proteomic assay or immunoassay. In some embodiments, the kit comprises an aptamer or antibody that specifically binds to SERPINF1, an aptamer or antibody that specifically binds to VEGFA, an aptamer or antibody that specifically binds to FLT1, an aptamer or antibody that specifically binds to VEGFB, and an aptamer or antibody that specifically binds to VEGFD.

In certain embodiments, the kit further comprises instructions for determining whether a subject has AMD.

In another aspect, a protein selected from the group consisting of serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) for use as a biomarker in diagnosing AMD is provided.

In another aspect, an in vitro method of diagnosing AMD is provided, the method comprising: obtaining an aqueous humor sample from an eye of the patient; and measuring levels of one or more biomarkers selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) in the aqueous humor sample, wherein increased levels of the one or more biomarkers selected from SERPINF1, VEGFA, FLT1, VEGFB, and VEGFD compared to reference value ranges for the levels of the one or more biomarkers in a control aqueous humor sample indicate that the patient has AMD.

In another aspect, a method of diagnosing and treating diabetic retinopathy in a patient is provided, the method comprising: obtaining an aqueous humor sample from an eye of the patient; measuring levels of one or more biomarkers selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein increased levels of the one or more biomarkers selected from SERPINF1, RARRES2, CFI, APP, C4A, and C4B compared to reference value ranges for the levels of the one or more biomarkers in a control aqueous humor sample indicate that the patient has diabetic retinopathy; and treating the patient for the diabetic retinopathy if the patient has a positive diagnosis for diabetic retinopathy.

In certain embodiments, the levels of at least two, at least three, or at least four biomarkers selected from SERPINF1, RARRES2, CFI, APP, C4A, and C4B are measured in the aqueous humor sample. In some embodiments, the levels of SERPINF1, RARRES2, CFI, APP, C4A, and C4B are measured in the aqueous humor sample.

In certain embodiments, treatment of the patient for the diabetic retinopathy comprises administering an anti-vascular endothelial growth factor (VEGF) agent or a steroid, or performing panretinal laser photocoagulation or a vitrectomy, or a combination thereof. In some embodiments, the anti-VEGF agent is bevacizumab, ranibizumab, sunitinib, sorafenib, axitinib, aflibercept, brolucizuma, faricimab, or pazopanib. In some embodiments, the steroid is triamcinolone acetonide, fluocinolone acetonide, or dexamethasone.

In certain embodiments, measuring the levels of the biomarkers comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot. In some embodiments, the levels of the biomarkers are measured using a multiplex aptamer array.

In certain embodiments, the subject has not yet developed clinical symptoms. In other embodiments, the subject has developed clinical symptoms.

In another aspect, a method of monitoring diabetic retinopathy in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein detection of increased levels of the one or more biomarkers selected from SERPINF1, RARRES2, CFI, APP, C4A, and C4B in the second aqueous humor sample compared to the first aqueous humor sample indicate that the patient is worsening, and wherein detection of decreased levels of the one or more biomarkers selected from SERPINF1, RARRES2, CFI, APP, C4A, and C4B in the second aqueous humor sample compared to the first aqueous humor sample indicate that the patient is improving.

In another aspect, a method of monitoring efficacy of a treatment of a patient for diabetic retinopathy is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from serpin family F member 1 (SERPINF1), retinoic acid receptor responder 2 (RARRES2), complement factor I (CFI), amyloid beta precursor protein (APP), complement C4A (C4A), and complement C4B (C4B); and evaluating the efficacy of the treatment, wherein detection of increased levels of the one or more biomarkers selected from SERPINF1, RARRES2, CFI, APP, C4A, and C4B in the second aqueous humor sample compared to the first aqueous humor sample indicate that the patient is worsening or not responding to the treatment, and detection of decreased levels of the one or more biomarkers selected from SERPINF1, RARRES2, CFI, APP, C4A, and C4B in the second aqueous humor sample compared to the first aqueous humor sample indicate that the patient is improving.

In certain embodiments, the method further comprises altering the treatment if the patient is worsening or not responding to the treatment.

In another aspect, a kit for diagnosing diabetic retinopathy is provided, the kit comprising agents for detecting at least 3 biomarkers selected from the group consisting of serpin family F member 1 (SERPINF1), retinoic acid receptor responder 2 (RARRES2), complement factor I (CFI), amyloid beta precursor protein (APP), complement C4A (C4A), and complement C4B (C4B). In certain embodiments, the kit comprises agents for detecting all of the SERPINF1, RARRES2, CFI, APP, C4A, and C4B biomarkers.

In certain embodiments, the kit further comprises reagents for performing an aptamer-based proteomic assay or immunoassay. In certain embodiments, the kit comprises an aptamer or antibody that specifically binds to SERPINF1, an aptamer or antibody that specifically binds to RARRES2, an aptamer or antibody that specifically binds to CFI, an aptamer or antibody that specifically binds to APP, an aptamer or antibody that specifically binds to C4A, and an aptamer or antibody that specifically binds to C4B.

In certain embodiments, the kit further comprises instructions for determining whether a subject has diabetic retinopathy.

In another aspect, a protein selected from the group consisting of serpin family F member 1 (SERPINF1), retinoic acid receptor responder 2 (RARRES2), complement factor I (CFI), amyloid beta precursor protein (APP), complement C4A (C4A), and complement C4B (C4B) for use as a biomarker in diagnosing diabetic retinopathy is provided.

In another aspect, an in vitro method of diagnosing diabetic retinopathy is provided, the method comprising: obtaining an aqueous humor sample from an eye of the patient; and measuring levels of one or more biomarkers selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein increased levels of the one or more biomarkers selected from SERPINF1, RARRES2, CFI, APP, C4A, and C4B compared to reference value ranges for the levels of the one or more biomarkers in a control aqueous humor sample indicate that the patient has diabetic retinopathy.

In another aspect, a method of diagnosing and treating proliferative vitreoretinopathy (PVR) in a patient is provided, the method comprising: obtaining an aqueous humor sample from an eye of the patient; measuring levels of one or more biomarkers selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein increased levels of the one or more biomarkers selected from IGFBP6, CCL15, CXCL12, VEGFA, and CCL23 compared to reference value ranges for the levels of the one or more biomarkers in a control aqueous humor sample indicate that the patient has PVR; and treating the patient for the PVR if the patient has a positive diagnosis for PVR.

In certain embodiments, the levels of at least two, at least three, or at least four biomarkers selected from IGFBP6, CCL15, CXCL12, VEGFA, and CCL23 are measured in the aqueous humor sample. In some embodiments, the levels of IGFBP6, CCL15, CXCL12, VEGFA, and CCL23 are measured in the aqueous humor sample.

In certain embodiments, treatment of the patient for the PVR comprises performing vitreous surgery, vitrectomy, membrane peeling, or retinotomy.

In certain embodiments, measuring the levels of the biomarkers comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot. In some embodiments, the levels of the biomarkers are measured using a multiplex aptamer array.

In certain embodiments, the subject has not yet developed clinical symptoms. In other embodiments, the subject has developed clinical symptoms.

In another aspect, a method of monitoring PVR in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein detection of increased levels of the one or more biomarkers selected from IGFBP6, CCL15, CXCL12, VEGFA, and CCL23 in the second aqueous humor sample compared to the first aqueous humor sample indicate that the patient is worsening, and wherein detection of decreased levels of the one or more biomarkers selected from IGFBP6, CCL15, CXCL12, VEGFA, and CCL23 in the second aqueous humor sample compared to the first aqueous humor sample indicate that the patient is improving.

In another aspect, a method of monitoring efficacy of a treatment of a patient for PVR is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23); and evaluating the efficacy of the treatment, wherein detection of increased levels of the one or more biomarkers selected from IGFBP6, CCL15, CXCL12, VEGFA, and CCL23 in the second aqueous humor sample compared to the first aqueous humor sample indicate that the patient is worsening or not responding to the treatment, and detection of decreased levels of the one or more biomarkers selected from IGFBP6, CCL15, CXCL12, VEGFA, and CCL23 in the second aqueous humor sample compared to the first aqueous humor sample indicate that the patient is improving.

In certain embodiments, the method further comprises altering the treatment if the patient is worsening or not responding to the treatment.

In another aspect, a kit for diagnosing PVR is provided, the kit comprising agents for detecting at least 3 biomarkers selected from the group consisting of insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23). In certain embodiments, the kit comprises agents for detecting all of the IGFBP6, CCL15, CXCL12, VEGFA, and CCL23 biomarkers.

In certain embodiments, the kit further comprises reagents for performing an aptamer-based proteomic assay or immunoassay. In some embodiments, the kit comprises an aptamer or antibody that specifically binds to IGFBP6, an aptamer or antibody that specifically binds to CCL15, an aptamer or antibody that specifically binds to CXCL12, an aptamer or antibody that specifically binds to VEGFA, and an aptamer or antibody that specifically binds to CCL23.

In certain embodiments, the kit further comprises instructions for determining whether a subject has PVR.

In another aspect, a protein selected from the group consisting of insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) for use as a biomarker in diagnosing PVR is provided.

In another aspect, an in vitro method of diagnosing PVR is provided, the method comprising: obtaining an aqueous humor sample from an eye of the patient; and measuring levels of one or more biomarkers selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein increased levels of the one or more biomarkers selected from IGFBP6, CCL15, CXCL12, VEGFA, and CCL23 compared to reference value ranges for the levels of the one or more biomarkers in a control aqueous humor sample indicate that the patient has PVR.

In another aspect, a method of diagnosing and treating melanoma in a patient is provided, the method comprising: obtaining an aqueous humor sample from an eye of the patient; measuring levels of one or more biomarkers selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein increased levels of the one or more biomarkers selected from FSTL1, ENPP2, ANG, MET, and HGF compared to reference value ranges for the levels of the one or more biomarkers in a control aqueous humor sample indicate that the patient has melanoma; and treating the patient for the melanoma if the patient has a positive diagnosis for melanoma.

In certain embodiments, the levels of at least two, at least three, or at least four biomarkers selected from FSTL1, ENPP2, ANG, MET, and HGF are measured in the aqueous humor sample. In some embodiments, the levels of FSTL1, ENPP2, ANG, MET, and HGF are measured in the aqueous humor sample.

In certain embodiments, treatment of the patient for the melanoma comprises performing surgery to excise the melanoma. In some embodiments, the treatment further comprises performing radiation therapy or administering interferon, interleukin-2 (IL-2), dacarbazine, vemurafenib, dabrafenib, trametinib, pembrolizumab, ipilimumab, tremelimumab, nivolumab/relatlimab, or imiquimod, or a combination thereof.

In certain embodiments, measuring the levels of the biomarkers comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot. In some embodiments, the levels of the biomarkers are measured using a multiplex aptamer array.

In certain embodiments, the subject has not yet developed clinical symptoms. In other embodiments, the subject has developed clinical symptoms.

In another aspect, a method of monitoring melanoma in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein detection of increased levels of the one or more biomarkers selected from FSTL1, ENPP2, ANG, MET, and HGF in the second aqueous humor sample compared to the first aqueous humor sample indicate that the patient is worsening, and wherein detection of decreased levels of the one or more biomarkers selected from FSTL1, ENPP2, ANG, MET, and HGF in the second aqueous humor sample compared to the first aqueous humor sample indicate that the patient is improving.

In another aspect, a method of monitoring efficacy of a treatment of a patient for melanoma is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF); and evaluating the efficacy of the treatment, wherein detection of increased levels of the one or more biomarkers selected from FSTL1, ENPP2, ANG, MET, and HGF in the second aqueous humor sample compared to the first aqueous humor sample indicate that the patient is worsening or not responding to the treatment, and detection of decreased levels of the one or more biomarkers selected from FSTL1, ENPP2, ANG, MET, and HGF in the second aqueous humor sample compared to the first aqueous humor sample indicate that the patient is improving.

In certain embodiments, the method further comprises altering the treatment if the patient is worsening or not responding to the treatment.

In certain embodiments, the melanoma is choroidal melanoma.

In another aspect, a kit for diagnosing melanoma is provided, the kit comprising agents for detecting at least 3 biomarkers selected from the group consisting of follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF). In certain embodiments, the kit comprises agents for detecting all of the FSTL1, ENPP2, ANG, MET, and HGF biomarkers.

In certain embodiments, the kit further comprises reagents for performing an aptamer-based proteomic assay or immunoassay. In some embodiments, the kit comprises an aptamer or antibody that specifically binds to FSTL1, an aptamer or antibody that specifically binds to ENPP2, an aptamer or antibody that specifically binds to ANG, an aptamer or antibody that specifically binds to MET, and an aptamer or antibody that specifically binds to HGF.

In certain embodiments, the kit further comprises instructions for determining whether a subject has melanoma.

In another aspect, a protein selected from the group consisting of follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) for use as a biomarker in diagnosing melanoma is provided.

In another aspect, an in vitro method of diagnosing melanoma is provided, the method comprising: obtaining an aqueous humor sample from an eye of the patient; and measuring levels of one or more biomarkers selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein increased levels of the one or more biomarkers selected from FSTL1, ENPP2, ANG, MET, and HGF compared to reference value ranges for the levels of the one or more biomarkers in a control aqueous humor sample indicate that the patient has melanoma.

In certain embodiments, the melanoma is choroidal melanoma.

In another aspect, a method of diagnosing and treating uveitis in a patient is provided, the method comprising: obtaining an aqueous humor sample from an eye of the patient; measuring levels of one or more biomarkers selected from retinoic acid receptor responder 2 (RARRES2), biotinidase (BTD), cystatin C (CST3), TIMP metallopeptidase inhibitor 2 (TIMP2), and prostaglandin D2 synthase (PTGDS) in the aqueous humor sample, wherein increased levels of the one or more biomarkers selected from RARRES2, BTD, CST3, TIMP2, and PTGDS compared to reference value ranges for the levels of the one or more biomarkers in a control aqueous humor sample indicate that the patient has uveitis; and treating the patient for the uveitis if the patient has a positive diagnosis for uveitis.

In certain embodiments, the levels of at least two, at least three, or at least four biomarkers selected from RARRES2, BTD, CST3, TIMP2, and PTGDS are measured in the aqueous humor sample. In some embodiments, the levels of RARRES2, BTD, CST3, TIMP2, and PTGDS are measured in the aqueous humor sample.

In certain embodiments, treatment of the patient for the uveitis comprises administering a glucocorticoid steroid, a cycloplegic agent, an antimetabolite, a T-cell inhibitor, an anti-tumor necrosis factor (TNF) agent, a biologic agent, an alkylating agent, an antibiotic for bacterial uveitis, an antiviral agent for viral uveitis, or an antifungal agent for fungal uveitis, or performing a vitrectomy, or a combination thereof.

Exemplary glucocorticoid steroids include, without limitation, prednisolone, methylprednisolone, iluvien, ozurdex, retisert, and triamcinolone. Exemplary T-cell inhibitors include, without limitation, calcineurin inhibitors such as cyclosporine, tacrolimus and voclosporin, and mTOR inhibitors such as everolimus and sirolimus. Exemplary antimetabolites include, without limitation, purine antagonists such as azathioprine, dihydrofolate reductase (DHFR) inhibitors such as methotrexate, and inosine monophosphate dehydrogenase (IMPDH) inhibitors such as mycophenolate mofetil. Exemplary anti-TNF agents include, without limitation, adalimumab, certolizumab, golimumab, infliximab, and etanercept. Exemplary biologic agents include, without limitation, efalizumab, rituximab, abatacept, alemtuzumab, anakinra, canakinumab, gevokizumab, daclizumab, tocilizumab, secukinumab, interferon α/β, fingolimod, aflibercept, bevacizumab, ranibizumab, and intravenous immunoglobulin (IVIG). Exemplary alkylating agents include, without limitation, chlorambucil and cyclophosphamide. Exemplary cycloplegic agents include, without limitation, atropine and homatropine. Exemplary antibiotics include, without limitation, cephalosporins, vancomycin, ceftazidime, amikacin, gentamycin, and moxifloxacin. Exemplary antiviral agents include, without limitation, ganciclovir, acyclovir, foscarnet, valacyclovir, and cidofivir. Exemplary antifungal agents include, without limitation, amphotericin B, voriconazole, caspofungin, and fluconazole.

In certain embodiments, measuring the levels of the biomarkers comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot. In some embodiments, the levels of the biomarkers are measured using a multiplex aptamer array.

In certain embodiments, the subject has not yet developed clinical symptoms. In other embodiments, the subject has developed clinical symptoms.

In another aspect, a method of monitoring uveitis in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from retinoic acid receptor responder 2 (RARRES2), biotinidase (BTD), cystatin C (CST3), TIMP metallopeptidase inhibitor 2 (TIMP2), and prostaglandin D2 synthase (PTGDS) in the aqueous humor sample, wherein detection of increased levels of the one or more biomarkers selected from RARRES2, BTD, CST3, TIMP2, and PTGDS in the second aqueous humor sample compared to the first aqueous humor sample indicate that the patient is worsening, and wherein detection of decreased levels of the one or more biomarkers selected from RARRES2, BTD, CST3, TIMP2, and PTGDS in the second aqueous humor sample compared to the first aqueous humor sample indicate that the patient is improving.

In another aspect, a method of monitoring efficacy of a treatment of a patient for uveitis is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from retinoic acid receptor responder 2 (RARRES2), biotinidase (BTD), cystatin C (CST3), TIMP metallopeptidase inhibitor 2 (TIMP2), and prostaglandin D2 synthase (PTGDS); and evaluating the efficacy of the treatment, wherein detection of increased levels of the one or more biomarkers selected from RARRES2, BTD, CST3, TIMP2, and PTGDS in the second aqueous humor sample compared to the first aqueous humor sample indicate that the patient is worsening or not responding to the treatment, and detection of decreased levels of the one or more biomarkers selected from RARRES2, BTD, CST3, TIMP2, and PTGDS in the second aqueous humor sample compared to the first aqueous humor sample indicate that the patient is improving.

In certain embodiments, the method further comprises altering the treatment if the patient is worsening or not responding to the treatment.

In another aspect, a kit for diagnosing uveitis is provided, the kit comprising agents for detecting at least 3 biomarkers selected from the group consisting of retinoic acid receptor responder 2 (RARRES2), biotinidase (BTD), cystatin C (CST3), TIMP metallopeptidase inhibitor 2 (TIMP2), and prostaglandin D2 synthase (PTGDS).

In certain embodiments, the kit comprises agents for detecting all of the RARRES2, BTD, CST3, TIMP2, and PTGDS biomarkers.

In certain embodiments, the kit further comprises reagents for performing an aptamer-based proteomic assay or immunoassay. In some embodiments, the kit comprises an aptamer or antibody that specifically binds to RARRES2, an aptamer or antibody that specifically binds to BTD, an aptamer or antibody that specifically binds to CST3, an aptamer or antibody that specifically binds to TIMP2, and an aptamer or antibody that specifically binds to PTGDS.

In certain embodiments, the kit further comprises instructions for determining whether a subject has uveitis.

In another aspect, a protein selected from the group consisting of retinoic acid receptor responder 2 (RARRES2), biotinidase (BTD), cystatin C (CST3), TIMP metallopeptidase inhibitor 2 (TIMP2), and prostaglandin D2 synthase (PTGDS) for use as a biomarker in diagnosing uveitis is provided.

In another aspect, an in vitro method of diagnosing uveitis is provided, the method comprising: obtaining an aqueous humor sample from an eye of the patient; and measuring levels of one or more biomarkers selected from cystatin-C(CST3) and metalloproteinase inhibitor 2 (TIMP2) in the aqueous humor sample, wherein increased levels of the one or more biomarkers selected from RARRES2, BTD, CST3, TIMP2, and PTGDS compared to reference value ranges for the levels of the one or more biomarkers in a control aqueous humor sample indicate that the patient has uveitis.

In another aspect, a method of diagnosing and treating neovascular inflammatory vitreoretinopathy (NIV) in a patient is provided, the method comprising: obtaining an aqueous humor sample from an eye of the patient; measuring levels of one or more biomarkers selected from serpin family C member 1 (SERPINC1), hemopexin (HPX), coagulation factor II (F2), complement C9 (C9), and complement C6 (C6) in the aqueous humor sample, wherein increased levels of the one or more biomarkers selected from SERPINC1, HPX, F2, C9, and C6 compared to reference value ranges for the levels of the one or more biomarkers in a control aqueous humor sample indicate that the patient has NIV; and treating the patient for the NIV if the patient has a positive diagnosis for NIV.

In certain embodiments, the levels of at least two, at least three, or at least four biomarkers selected from SERPINC1, HPX, F2, C9, and C6 are measured in the aqueous humor sample. In some embodiments, the levels of SERPINC1, HPX, F2, C9, and C6 are measured in the aqueous humor sample.

In certain embodiments, treatment of the patient for the neovascular inflammatory vitreoretinopathy comprises administering fluocinolone acetonide, dexamethasone, or bevacizumab, or performing panretinal scatter photocoagulation (PRP), vitrectomy, trabeculectomy, or a combination thereof.

In certain embodiments, measuring the levels of the biomarkers comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot. In some embodiments, the levels of the biomarkers are measured using a multiplex aptamer array.

In certain embodiments, the subject has not yet developed clinical symptoms. In other embodiments, the subject has developed clinical symptoms.

In another aspect, a method of monitoring NIV in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from serpin family C member 1 (SERPINC1), hemopexin (HPX), coagulation factor II (F2), complement C9 (C9), and complement C6 (C6) in the aqueous humor sample, wherein detection of increased levels of the one or more biomarkers selected from SERPINC1, HPX, F2, C9, and C6 in the second aqueous humor sample compared to the first aqueous humor sample indicate that the patient is worsening, and wherein detection of decreased levels of the one or more biomarkers selected from SERPINC1, HPX, F2, C9, and C6 in the second aqueous humor sample compared to the first aqueous humor sample indicate that the patient is improving.

In another aspect, a method of monitoring efficacy of a treatment of a patient for NIV is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from serpin family C member 1 (SERPINC1), hemopexin (HPX), coagulation factor II (F2), complement C9 (C9), and complement C6 (C6); and evaluating the efficacy of the treatment, wherein detection of increased levels of the one or more biomarkers selected from SERPINC1, HPX, F2, C9, and C6 in the second aqueous humor sample compared to the first aqueous humor sample indicate that the patient is worsening or not responding to the treatment, and detection of decreased levels of the one or more biomarkers selected from SERPINC1, HPX, F2, C9, and C6 in the second aqueous humor sample compared to the first aqueous humor sample indicate that the patient is improving.

In certain embodiments, the method further comprises altering the treatment if the patient is worsening or not responding to the treatment.

In another aspect, a kit for diagnosing NIV is provided, the kit comprising agents for detecting at least 3 biomarkers selected from the group consisting of serpin family C member 1 (SERPINC1), hemopexin (HPX), coagulation factor II (F2), complement C9 (C9), and complement C6 (C6). In certain embodiments, the kit comprises agents for detecting all of the SERPINC1, HPX, F2, C9, and C6 biomarkers.

In certain embodiments, the kit further comprises reagents for performing an aptamer-based proteomic assay or immunoassay. In some embodiments, the kit comprises an aptamer or antibody that specifically binds to SERPINC1, an aptamer or antibody that specifically binds to HPX, an aptamer or antibody that specifically binds to F2, an aptamer or antibody that specifically binds to C9, and an aptamer or antibody that specifically binds to C6.

In certain embodiments, the kit further comprises instructions for determining whether a subject has NIV.

In another aspect, a protein selected from the group consisting of serpin family C member 1 (SERPINC1), hemopexin (HPX), coagulation factor II (F2), complement C9 (C9), and complement C6 (C6) for use as a biomarker in diagnosing NIV is provided.

In another aspect, an in vitro method of diagnosing neovascular inflammatory vitreoretinopathy (NIV) is provided, the method comprising: obtaining an aqueous humor sample from an eye of the patient; and measuring levels of one or more biomarkers selected from serpin family C member 1 (SERPINC1), hemopexin (HPX), coagulation factor II (F2), complement C9 (C9), and complement C6 (C6) in the aqueous humor sample, wherein increased levels of the one or more biomarkers selected from SERPINC1, HPX, F2, C9, and C6 compared to reference value ranges for the levels of the one or more biomarkers in a control aqueous humor sample indicate that the patient has NIV.

### BRIEF DESCRIPTION OF THE DRAWINGS

FIGS. 1A-1F. Liquid chromatography-mass spectrometry (LC-MS) detects highest number of unique proteins in undepleted aqueous humor samples. (FIG. 1A) Influence of protein depletion method on number of unique proteins detected by LC-MS in aqueous humor (AH). Left panel: The height of each bar represents the mean number of proteins in 3 samples, the error bar corresponds to the standard deviation. Right panel: Overlap of proteins detected by LC-MS after albumin depletion (blue), albumin and immunoglobulin (IG) depletion (red) as well as top 14 depletion (yellow) each compared to undepleted samples (green). Only proteins which were detected in each of the 3 samples were considered. (FIG. 1B) Impact of protein depletion on off-target proteins. The proteins which are targeted by the largest depletion method (top14) are shown. The height of each bar represents the mean spectral count (log 2 scaled). Each dot represents one sample. For simplicity, only the 3 most abundant immunoglobulins are visualized. (FIGS. 1C-1E) Visualization of differentially expressed proteins between each depletion method and undepleted samples. The log 2-scaled mean spectral count for each protein between undepleted samples (x-axis) and each depletion method (y-axis) is shown. Proteins that were significantly more abundant in non-depleted samples are shown in green. Proteins that were increased after albumin depletion (FIG. 1C), albumin and IG depletion (FIG. 1D) as well as top 14 depletion (FIG. 1E) are shown in blue, red or yellow, respectively. The top differentially expressed proteins are labeled. (FIG. 1F) Impact of protein depletion method on detection of low abundant proteins with less than 10 mean spectral counts in each group. The z-score represents the deviation from a protein's mean abundance in standard deviation units. Abbreviations: undepl: undepleted, Alb+IG: albumin and immunoglobulin depletion, Top14: top 14 depletion, Alb: albumin depletion.

FIGS. 2A-2D. Aptamer-based assay detects large number of previously unknown aqueous humor proteins and suggests age-related proteomic signatures. (FIG. 2A) Heatmap visualizing protein intensities of 2,696 proteins which were above the limit of detection in all 8 samples (upper panel), 2,245 proteins detected in 1 to 7 samples (middle panel) and 1,402 proteins which are included in the aptamer-based assay but were not expressed in any aqueous humor sample (lower panel). Each row represents one protein and each column represents one sample. Basic demographic data are shown at the top of the heatmap, indicating age-dependent alterations of the aqueous humor proteome. (FIG. 2B) Comparison of aqueous humor (AH) proteins identified by aptamer-based assay to previously reported AH proteins (mass spectrometry) from control as well as diseased samples (age-related macular degeneration, diabetic retinopathy, Coat's disease, glaucoma and uveal melanoma). Proteins detected in control AH by the aptamer-based assay are shown in green. Proteins reported in the literature in control and diseased AH are shown in grey or blue, respectively. (FIG. 2C) Age-dependent proteomics signatures in AH. MMP10 and NRP1 were among the proteins which were significantly correlated with patient's age (p<0.05 in limma model, the squared Pearson correlation coefficient is shown). Each dot represents one sample. (FIG. 2D): SRF target proteins are downregulated in AH with aging. Serum response factor (SRF) has recently been shown to mediate rejuvenation of aged brains by cerebrospinal fluid transferred from young to old mice. The Pearson correlation coefficient between protein intensity in AH and patient's age is shown in the boxplot. Each dot represents one of the 22 SRF target proteins detected in AH. Correlation with age for three of these proteins (Pearson p<0.05) is visualized on the right. The squared Pearson correlation coefficient is shown. Each dot represents one sample.

FIGS. 3A-3D. Functionally grouped network analysis indicates that axon guidance molecules may play a key role in AH. (FIG. 3A): Functionally grouped network of enriched Gene ontology (GO)/pathway terms in the aqueous humor proteome. Enriched terms are visualized as nodes being linked based on their kappa score (20.3), which indicates the similarity of the proteins linked to them. The node size represents the term enrichment significance. The pie charts visualize the percentage of proteins previously also detected by LC-MS (grey) and those proteins which were exclusively detected by the aptamer-based assay (colored). The colors correspond to functionally related groups. (FIG. 3B) STRING interaction network based on the proteins of the axon guidance cluster from (FIG. 3A). Proteins which were exclusively detected by the aptamer-based assay are shown in purple. Proteins which were already known in the AH are visualized in grey. (FIG. 3C) Proteins involved in key networks in AH (FIG. 3A) compared to blood. For each subnetwork from (FIG. 3A), the percentage of proteins being more abundant in AH are shown in yellow. Proteins with higher intensities in blood are visualized in red and proteins with similar abundance in AH and blood are shown in grey. Striped areas represent proteins, that were newly identified using the aptamer-based assay. (FIG. 3D) AH proteins from each network are enriched in AH compared to blood. The height of the bars corresponds to the percentile of protein intensity in AH or blood.

FIGS. 4A-4B. Aptamer-based assay highlights aqueous humor as a compelling diagnostic platform for vitreoretinal diseases. (FIG. 4A): Proteomic biomarkers previously identified in the vitreous of patients with different vitreoretinal diseases are detectable in the aqueous humor using the aptamer-based assay. Proteins which were detected in each or in at least one sample are colored in green or blue, respectively. Proteins not detected are shown in yellow and those not included in the aptamer-based assay but previously detected in the diseased vitreous are shown in grey. The absolute numbers of proteins are shown within the bars. AH: aqueous humor, AMD: age-related macular degeneration, DR: diabetic retinopathy, PVR: proliferative vitreoretinopathy, melanoma: choroidal melanoma, NIV: Neovascular Inflammatory Vitreoretinopathy. (FIG. 4B): protein intensities of the top 5 proteins for each disease as well as five retina- and RPE-specific proteins indicating an exchange of proteins between AH and vitreous.

### DETAILED DESCRIPTION OF THE INVENTION

Compositions, methods, and kits are provided for diagnosing vitreoretinal diseases and age-related pathologies. In particular, aqueous humor biomarkers have been identified that correlate with biological aging and age-related pathologies and morbidity. The use of such biomarkers may allow earlier intervention in treatment of aging-related diseases. In addition, the inventors have discovered that protein exchange occurs between the vitreous and aqueous humor of the eye, which enables monitoring of biomarkers of various vitreoretinal diseases by measuring levels of biomarkers in the aqueous humor.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a biomarker” includes a plurality of such biomarkers and reference to “the polypeptide” includes reference to one or more polypeptides and equivalents thereof, e.g., peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Biomarkers. The term “biomarker” as used herein refers to a compound, such as a protein, a polypeptide, a peptide, a mRNA, a metabolite, or a metabolic byproduct which is differentially expressed or present at different concentrations, levels or frequencies in one sample compared to another, such as an aqueous humor sample from patients who have a vitreoretinal disease, melanoma, or risk of age-related pathology and morbidity compared to an aqueous humor sample from healthy control subjects (i.e., subjects not having a vitreoretinal disease or melanoma, or younger healthy subjects not at risk of age-related pathology and morbidity). Biomarkers include, but are not limited to, biomarkers for predicting biological age and determining risk of age-related pathology such as stromelysin-2 (MMP10), neuropilin-1 (NRP1), semaphorin 3C (SEMA3C), hes family bHLH transcription factor 5 (HES5), and fibroblast growth factor receptor like 1 (FGFRL1); biomarkers for diagnosing age-related macular degeneration (AMD) such as serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD); biomarkers for diagnosing diabetic retinopathy such as serpin family F member 1 (SERPINF1), retinoic acid receptor responder 2 (RARRES2), complement factor I (CFI), amyloid beta precursor protein (APP), complement C4A (C4A), and complement C4B (C4B); biomarkers for diagnosing proliferative vitreoretinopathy (PVR) such as insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23); biomarkers for diagnosing melanoma such as follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF); biomarkers for diagnosing uveitis such as retinoic acid receptor responder 2 (RARRES2), biotinidase (BTD), cystatin C (CST3), TIMP metallopeptidase inhibitor 2 (TIMP2), and prostaglandin D2 synthase (PTGDS); and biomarkers for diagnosing neovascular inflammatory vitreoretinopathy (NIV) such as serpin family C member 1 (SERPINC1), hemopexin (HPX), coagulation factor II (F2), complement C9 (C9), and complement C6 (C6).

In some embodiments, the concentration or level of a biomarker is determined before and after the administration of a treatment to a patient. The treatment may comprise, for example, without limitation, administering a corticosteroid or a vascular endothelial growth factor inhibitor or performing laser surgery or a vitrectomy. The degree of change in the concentration or level of a biomarker, or lack thereof, is interpreted as an indication of whether the treatment has the desired effect (e.g., preventing or reducing damage to the retina and loss of vision). In other words, the concentration or level of a biomarker is determined before and after the administration of the treatment to an individual, and the degree of change, or lack thereof, in the level is interpreted as an indication of whether the individual is “responsive” to the treatment.

A “reference level” or “reference value” of a biomarker means a level of the biomarker that is indicative of a particular biological age, disease state (e.g., melanoma or a vitreoretinal disease such as age-related macular degeneration, diabetic retinopathy, proliferative vitreoretinopathy, uveitis, or neovascular inflammatory vitreoretinopathy), phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular biological age or disease state or phenotype. A “negative” reference level of a biomarker means a level that is indicative of a lack of a particular biological age or disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in aqueous humor samples (e.g., aptamer-based assays, immunoassays (e.g., ELISA), mass spectrometry (e.g., LC-MS, GC-MS), tandem mass spectrometry, NMR, biochemical or enzymatic assays, PCR, microarray analysis, etc.), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to a “biological age” biomarker expression profile, an “age-related macular degeneration” biomarker expression profile, a “diabetic retinopathy” biomarker expression profile, a “proliferative vitreoretinopathy” biomarker expression profile, a “uveitis” biomarker expression profile, a “neovascular inflammatory vitreoretinopathy” biomarker expression profile, or a “melanoma” biomarker expression profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Aqueous humor sample. The term “aqueous humor sample” with respect to an individual encompasses samples of ocular fluid secreted from the ciliary body of the eye. Aqueous humor is located in the anterior and posterior chambers of the eye. Aqueous humor samples can be obtained by any suitable method such as by liquid biopsy or surgically. The definition also includes samples that have been manipulated in any way after their procurement, such as by treatment with reagents; washed; or enriched for particular types of molecules, e.g., proteins, peptides, etc.

Obtaining and assaying a sample. The term “assaying” is used herein to include the physical steps of manipulating an aqueous humor sample to generate data related to the aqueous humor sample. As will be readily understood by one of ordinary skill in the art, an aqueous humor sample must be “obtained” prior to assaying the sample. Thus, the term “assaying” implies that the sample has been obtained. The terms “obtained” or “obtaining” as used herein encompass the act of receiving an extracted or isolated aqueous humor sample. For example, a testing facility can “obtain” an aqueous humor sample in the mail (or via delivery, etc.) prior to assaying the sample. In some such cases, the aqueous humor sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of an aqueous humor sample from a subject. Accordingly, an aqueous humor sample can be isolated from a subject (and thus “obtained”) by the same person or same entity that subsequently assays the sample. When an aqueous humor sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating an aqueous humor sample.

In some embodiments, the step of obtaining comprises the step of isolating an aqueous humor sample (e.g., a pre-treatment aqueous humor sample, a post-treatment aqueous humor sample, etc.). Methods and protocols for isolating various aqueous humor samples will be known to one of ordinary skill in the art and any convenient method may be used to isolate an aqueous humor sample.

It will be understood by one of ordinary skill in the art that in some cases, it is convenient to wait until multiple samples have been obtained prior to assaying the samples. Accordingly, in some cases an isolated aqueous humor sample is stored until all appropriate samples have been obtained. One of ordinary skill in the art will understand how to appropriately store a variety of different types of aqueous humor samples and any convenient method of storage may be used (e.g., refrigeration) that is appropriate for the particular aqueous humor sample. In some embodiments, a pre-treatment aqueous humor sample is assayed prior to obtaining a post-treatment aqueous humor sample. In some cases, a pre-treatment aqueous humor sample and a post-treatment aqueous humor sample are assayed in parallel. In some cases, multiple different post-treatment aqueous humor samples and/or a pre-treatment aqueous humor sample are assayed in parallel. In some cases, aqueous humor samples are processed immediately or as soon as possible after they are obtained.

In some embodiments, the concentration (i.e., “level”), or expression level of a gene product, which may be a protein, peptide, etc., (which will be referenced herein as a biomarker), in an aqueous humor sample is measured (i.e., “determined”). By “expression level” (or “level”) it is meant the level of gene product (e.g., the absolute and/or normalized value determined for the RNA expression level of a biomarker or for the expression level of the encoded polypeptide, or the concentration of the protein in an aqueous humor sample). The term “gene product” or “expression product” are used herein to refer to the RNA transcription products (RNA transcripts, e.g., mRNA, an unspliced RNA, a splice variant mRNA, and/or a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple aqueous humor samples from the same individual (e.g., aqueous humor samples taken at different points in time from the same individual). This is because the units cancel when calculating a fold-change (i.e., determining a ratio) in the expression level from one aqueous humor sample to the next (e.g., aqueous humor samples taken at different points in time from the same individual).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for the biomarker expression level. Any convenient protocol for evaluating RNA levels may be employed wherein the level of one or more RNAs in the assayed sample is determined.

For measuring protein levels, the amount or level of a protein in the aqueous humor sample is determined. In some cases, the protein comprises a post-translational modification (e.g., phosphorylation, glycosylation) associated with regulation of activity of the protein such as by a signaling cascade, wherein the modified protein is the biomarker, and the amount of the modified protein is therefore measured. In some embodiments, an extracellular protein level is measured. For example, in some cases, the protein (i.e., polypeptide) being measured is a secreted protein, and the concentration can be measured in aqueous humor. In some embodiments, concentration is a relative value measured by comparing the level of one protein relative to another protein. In other embodiments the concentration is an absolute measurement of weight/volume or weight/weight.

In some instances, the concentration of one or more additional proteins may also be measured, and biomarker concentration compared to the level of the one or more additional proteins to provide a normalized value for the biomarker concentration. Any convenient protocol for evaluating protein levels may be employed wherein the level of one or more proteins in the assayed sample is determined.

While a variety of different methods of assaying protein levels are known to one of ordinary skill in the art, and any convenient method may be used, two representative and convenient techniques for assaying protein levels include aptamer-based assays and antibody-based methods such as the enzyme-linked immunosorbent assay (ELISA). Aptamer-based assays use aptamers comprising single-stranded oligonucleotides that bind specifically to biomarker proteins of interest. Either high affinity RNA aptamers or DNA aptamers with specificity for a protein of interest may be used. Functional groups that mimic amino acid side-chains may be added to aptamers to confer protein-like properties to improve binding affinity to a protein of interest. Aptamers that bind specifically and with high affinity to a biomarker protein of interest can be selected from large libraries of aptamers having randomized sequences using Systematic Evolution of Ligands by EXponential enrichment (SELEX). The aptamers may be designed with unique nucleotide sequences recognizable by specific hybridization probes for capture on a hybridization array for multiplexed detection of biomarkers (see, e.g., Gold et al. (2010) Aptamer-Based Multiplexed Proteomic Technology for Biomarker Discovery. PLoS ONE 5(12):e15004; herein incorporated by reference in its entirety.

In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, other methods for measuring the levels of one or more proteins in a sample may be employed. Representative exemplary methods include but are not limited to antibody-based methods (e.g., immunofluorescence assay, radioimmunoassay, immunoprecipitation, Western blotting, proteomic arrays, xMAP microsphere technology (e.g., Luminex technology), immunohistochemistry, flow cytometry, and the like) as well as non-antibody-based methods (e.g., mass spectrometry or tandem mass spectrometry).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

### Additional Terms

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with melanoma or a vitreoretinal disease) as well as those in which prevention is desired, those with a genetic predisposition to developing melanoma or a vitreoretinal disease, those with increased susceptibility to melanoma or a vitreoretinal disease, those suspected of having melanoma or a vitreoretinal disease, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “about,” particularly in reference to a given quantity, is meant to encompass deviations of plus or minus five percent.

The terms “individual”, “subject”, and “patient”, are used interchangeably herein and refer to any mammalian subject for whom diagnosis, treatment, or therapy is desired, particularly humans. Mammals include human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows. In some cases, the methods of the invention find use in experimental animals, in veterinary application, and in the development of animal models for disease, including, but not limited to, rodents including mice, rats, and hamsters; primates, and transgenic animals.

“Substantially purified” generally refers to isolation of a component such as a substance (compound, drug, inhibitor, metabolite, nucleic acid, polynucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BiTE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., biomarker) refers to a binding reaction that is determinative of the presence of the antigen in a heterogeneous population of proteins and other biologics. Thus, under designated immunoassay conditions, the specified antibodies bind to a particular antigen at least two times over the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of aqueous humor sample(s) was used and/or how it was used, the technique used to assay the sample, the results of the assay (e.g., the level of the biomarker as measured, and/or the fold-change of a biomarker level over time, or in a post-treatment assay compared to a pre-treatment assay), the assessment as to whether the individual is determined to have melanoma or a vitreoretinal disease, the predicted biological age of the individual and risk of age-related pathology and morbidity, a recommendation for additional screening for pathology, a recommendation for treatment, and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Biomarkers and Diagnostic Methods

Biomarkers that can be used in the practice of the subject methods include, without limitation, biomarkers for predicting biological age and determining risk of age-related pathology such as stromelysin-2 (MMP10), neuropilin-1 (NRP1), semaphorin 3C (SEMA3C), hes family bHLH transcription factor 5 (HES5), and fibroblast growth factor receptor like 1 (FGFRL1); biomarkers for diagnosing age-related macular degeneration (AMD) such as serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD); biomarkers for diagnosing diabetic retinopathy such as serpin family F member 1 (SERPINF1), retinoic acid receptor responder 2 (RARRES2), complement factor I (CFI), amyloid beta precursor protein (APP), complement C4A (C4A), and complement C4B (C4B); biomarkers for diagnosing proliferative vitreoretinopathy (PVR) such as insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23); biomarkers for diagnosing melanoma such as follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF); biomarkers for diagnosing uveitis such as retinoic acid receptor responder 2 (RARRES2), biotinidase (BTD), cystatin C (CST3), TIMP metallopeptidase inhibitor 2 (TIMP2), and prostaglandin D2 synthase (PTGDS); and biomarkers for diagnosing neovascular inflammatory vitreoretinopathy (NIV) such as serpin family C member 1 (SERPINC1), hemopexin (HPX), coagulation factor II (F2), complement C9 (C9), and complement C6 (C6).

In certain embodiments, a panel of biomarkers is provided. Biomarker panels of any size can be used in the practice of the subject methods. Biomarker panels typically comprise at least 3 biomarkers and up to 20 biomarkers, including any number of biomarkers in between, such as 3, 4, 5, 6, 7, 8, 9, 10, 11, 12, 13, 14, 15, 16, 17, 18, 19, or 20 biomarkers. In certain embodiments, a biomarker panel comprising at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or at least 20, or more biomarkers. Although smaller biomarker panels are usually more economical, larger biomarker panels (i.e., greater than 20 biomarkers) have the advantage of providing more detailed information and can also be used in the practice of the subject methods.

In some embodiments, a biomarker panel for predicting biological age and/or determining risk of age-related pathology comprises or consists of at least two, at least three, or at least four biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1). In some embodiments, a biomarker panel for predicting biological age and determining risk of age-related pathology comprises or consists of stromelysin-2 (MMP10), neuropilin-1 (NRP1), semaphorin 3C (SEMA3C), hes family bHLH transcription factor 5 (HES5), and fibroblast growth factor receptor like 1 (FGFRL1).

In some embodiments, a biomarker panel for diagnosing age-related macular degeneration (AMD) comprises or consists of at least two, at least three, or at least four biomarkers selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD). In some embodiments, a biomarker panel for diagnosing age-related macular degeneration (AMD) comprises or consists of serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD).

In some embodiments, a biomarker panel for diagnosing diabetic retinopathy comprises or consists of at least two, at least three, or at least four biomarkers selected from serpin family F member 1 (SERPINF1), retinoic acid receptor responder 2 (RARRES2), complement factor I (CFI), amyloid beta precursor protein (APP), complement C4A (C4A), and complement C4B (C4B). In some embodiments, a biomarker panel for diagnosing diabetic retinopathy comprises or consists of serpin family F member 1 (SERPINF1), retinoic acid receptor responder 2 (RARRES2), complement factor I (CFI), amyloid beta precursor protein (APP), complement C4A (C4A), and complement C4B (C4B).

In some embodiments, a biomarker panel for diagnosing proliferative vitreoretinopathy (PVR) comprises or consists of at least two, at least three, or at least four biomarkers selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23). In some embodiments, a biomarker panel for diagnosing proliferative vitreoretinopathy (PVR) comprises or consists of insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23).

In some embodiments, a biomarker panel for diagnosing uveitis comprises or consists of at least two, at least three, or at least four biomarkers selected from retinoic acid receptor responder 2 (RARRES2), biotinidase (BTD), cystatin C (CST3), TIMP metallopeptidase inhibitor 2 (TIMP2), and prostaglandin D2 synthase (PTGDS). In some embodiments, a biomarker panel for diagnosing uveitis comprises or consists of retinoic acid receptor responder 2 (RARRES2), biotinidase (BTD), cystatin C (CST3), TIMP metallopeptidase inhibitor 2 (TIMP2), and prostaglandin D2 synthase (PTGDS).

In some embodiments, a biomarker panel for diagnosing neovascular inflammatory vitreoretinopathy (NIV) comprises or consists of at least two, at least three, or at least four biomarkers selected from serpin family C member 1 (SERPINC1), hemopexin (HPX), coagulation factor II (F2), complement C9 (C9), and complement C6 (C6). In some embodiments, a biomarker panel for diagnosing neovascular inflammatory vitreoretinopathy (NIV) comprises or consists of serpin family C member 1 (SERPINC1), hemopexin (HPX), coagulation factor II (F2), complement C9 (C9), and complement C6 (C6).

In some embodiments, a biomarker panel for diagnosing melanoma comprises or consists of at least two, at least three, or at least four biomarkers selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF). In some embodiments, a biomarker panel for diagnosing melanoma comprises or consists of follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF).

An aqueous humor sample comprising the expressed biomarkers is obtained from the subject. The sample is taken from the aqueous humor (i.e., ocular fluid secreted from the ciliary body found in the anterior and posterior chambers of the eye). A “control” sample, as used herein, refers to an aqueous humor sample obtained from a normal or healthy subject (i.e., subject not having a vitreoretinal disease or melanoma, or younger healthy subject not at risk of age-related pathology and morbidity). An aqueous humor sample can be obtained from a subject by conventional techniques. For example, aqueous humor samples can be obtained by any suitable method such as liquid biopsy or surgically according to methods well known in the art.

When analyzing the levels of biomarkers in an aqueous humor sample from a subject, the reference value ranges used for comparison can represent the levels of one or more biomarkers in an aqueous humor sample from one or more subjects without disease (i.e., normal or healthy control). In some embodiments, the reference value ranges used for comparison can represent the levels of one or more biomarkers in aqueous humor samples from one or more subjects at certain biological ages to allow the biological age of an individual to be determined. Alternatively, the reference values can represent the levels of one or more biomarkers from one or more subjects with a disease (e.g., vitreoretinal disease or melanoma), wherein similarity to the reference value ranges indicates the subject has the disease. More specifically, the reference value ranges can represent the levels of one or more biomarkers from one or more subjects with age-related macular degeneration (an “age-related macular degeneration” biomarker expression profile), diabetic retinopathy (a “diabetic retinopathy” biomarker expression profile), proliferative vitreoretinopathy (a “proliferative vitreoretinopathy” biomarker expression profile), uveitis (a “uveitis” biomarker expression profile), neovascular inflammatory vitreoretinopathy (a “neovascular inflammatory vitreoretinopathy” biomarker expression profile), or melanoma (a “melanoma” biomarker expression profile).

Accordingly, in one aspect, a method of predicting biological age and determining risk of age-related pathology and morbidity in a patient is provided, the method comprising: obtaining an aqueous humor sample from an eye of the patient; and measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein decreased levels of the one or more biomarkers selected from MMP10, NRP1, SEMA3C, HES5, and FGFRL1 compared to reference value ranges for the levels of the one or more biomarkers in a control aqueous humor sample indicate that the patient has a risk of age-related pathology and morbidity. In certain embodiments, the method further comprises increasing screening of the patient for an aging-related disease if the patient is identified as having a risk of age-related pathology and morbidity. The subject methods may allow earlier treatment of aging-related diseases to delay disease progression and earlier medical intervention to prolong the life and/or improve the health of the patient.

In certain embodiments, a method of monitoring biological aging of an eye in a patient is provided, the method comprising: obtaining an aqueous humor sample from an eye of the patient; and measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein levels of the one or more biomarkers selected from MMP10, NRP1, SEMA3C, HES5, and FGFRL1 are correlated with biological age of the eye. In certain embodiments, the levels of at least two, at least three, or at least four biomarkers selected from MMP10, NRP1, SEMA3C, HES5, and FGFRL1 are measured in the aqueous humor sample. In some embodiments, the levels of MMP10, NRP1, SEMA3C, HES5, and FGFRL1 are measured in the aqueous humor sample. In certain embodiments, the method further comprises administering a treatment for an age-related eye disease to the patient if the patient is identified as having the age-related eye disease. Age-related eye diseases include, but are not limited to, age-related macular degeneration, cataracts, diabetic retinopathy, glaucoma, uveitis, neovascular inflammatory vitreoretinopathy, eye melanoma (e.g., choroidal melanoma), dry eye, retinal detachment, presbyopia, keratoconjunctivitis sicca, and epiphora.

Additional biomarkers may be used in diagnostic tests to distinguish among age-related eye diseases. Accordingly, in another aspect, a method of diagnosing age-related macular degeneration (AMD) in a patient is provided, the method comprising: obtaining an aqueous humor sample from an eye of the patient; and measuring levels of one or more biomarkers selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) in the aqueous humor sample, wherein increased levels of the one or more biomarkers selected from SERPINF1, VEGFA, FLT1, VEGFB, and VEGFD compared to reference value ranges for the levels of the one or more biomarkers in a control aqueous humor sample indicate that the patient has AMD. The method may further comprise determining an appropriate treatment regimen for a patient and, in particular, whether a patient should be treated for AMD. For example, a patient is selected for treatment for AMD if the patient has a positive diagnosis for AMD based on a biomarker expression profile, as described herein. The treatment for AMD may comprise, for example, administering vitamin C, zinc, vitamin E, copper, beta-carotene, lutein, zeaxanthin, ranibizumab, aflibercept, brolucizumab, or faricimab, or a combination thereof to the patient.

In some embodiments, the methods described herein are used for monitoring AMD in a subject. For example, a first aqueous humor sample can be obtained from the patient at a first time point and a second aqueous humor sample can be obtained from the subject at a second (later) time point. In one embodiment, AMD is monitored in the patient by measuring levels of one or more biomarkers in the first aqueous humor sample and the second aqueous humor sample, wherein the biomarkers are selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) in the aqueous humor sample, wherein detection of increased levels of the one or more biomarkers selected from SERPINF1, VEGFA, FLT1, VEGFB, and VEGFD in the second aqueous humor sample compared to the first aqueous humor sample indicate that the patient is worsening, and wherein detection of decreased levels of the one or more biomarkers selected from SERPINF1, VEGFA, FLT1, VEGFB, and VEGFD in the second aqueous humor sample compared to the first aqueous humor sample indicate that the patient is improving.

The subject methods may also be used for assaying pre-treatment and post-treatment aqueous humor samples obtained from an individual to determine whether the individual is responsive or not responsive to a treatment. For example, a first aqueous humor sample can be obtained from a subject before the subject undergoes the therapy, and a second aqueous humor sample can be obtained from the subject after the subject undergoes the therapy. In one embodiment, the efficacy of a treatment of a patient for AMD is monitored by measuring one or more biomarkers selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD); and evaluating the efficacy of the treatment, wherein detection of increased levels of the one or more biomarkers selected from SERPINF1, VEGFA, FLT1, VEGFB, and VEGFD in the second aqueous humor sample compared to the first aqueous humor sample indicate that the patient is worsening or not responding to the treatment, and detection of decreased levels of the one or more biomarkers selected from SERPINF1, VEGFA, FLT1, VEGFB, and VEGFD in the second aqueous humor sample compared to the first aqueous humor sample indicate that the patient is improving. In certain embodiments, the method further comprises altering the treatment if the patient is worsening or not responding to the treatment.

In another aspect, a method of diagnosing diabetic retinopathy in a patient is provided, the method comprising: obtaining an aqueous humor sample from an eye of the patient; and measuring levels of one or more biomarkers selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein increased levels of the one or more biomarkers selected from SERPINF1, RARRES2, CFI, APP, C4A, and C4B compared to reference value ranges for the levels of the one or more biomarkers in a control aqueous humor sample indicate that the patient has diabetic retinopathy. The method may further comprise determining an appropriate treatment regimen for a patient and, in particular, whether a patient should be treated for diabetic retinopathy. For example, a patient is selected for treatment for diabetic retinopathy if the patient has a positive diagnosis for diabetic retinopathy based on a biomarker expression profile, as described herein. The treatment for diabetic retinopathy may comprise, for example, administering an anti-vascular endothelial growth factor (VEGF) agent or a steroid, or performing panretinal laser photocoagulation or a vitrectomy, or a combination thereof. Exemplary anti-VEGF agent include, without limitation, bevacizumab, ranibizumab, sunitinib, sorafenib, axitinib, aflibercept, brolucizuma, faricimab, and pazopanib. Exemplary steroids include, without limitation, triamcinolone acetonide, fluocinolone acetonide, and dexamethasone.

In some embodiments, the methods described herein are used for monitoring diabetic retinopathy in a subject. For example, a first aqueous humor sample can be obtained from the patient at a first time point and a second aqueous humor sample can be obtained from the subject at a second (later) time point. In one embodiment, diabetic retinopathy is monitored in the patient by measuring levels of one or more biomarkers in the first aqueous humor sample and the second aqueous humor sample, wherein the biomarkers are selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein detection of increased levels of the one or more biomarkers selected from SERPINF1, RARRES2, CFI, APP, C4A, and C4B in the second aqueous humor sample compared to the first aqueous humor sample indicate that the patient is worsening, and wherein detection of decreased levels of the one or more biomarkers selected from SERPINF1, RARRES2, CFI, APP, C4A, and C4B in the second aqueous humor sample compared to the first aqueous humor sample indicate that the patient is improving.

The subject methods may also be used for assaying pre-treatment and post-treatment aqueous humor samples obtained from an individual to determine whether the individual is responsive or not responsive to a treatment. For example, a first aqueous humor sample can be obtained from a subject before the subject undergoes the therapy, and a second aqueous humor sample can be obtained from the subject after the subject undergoes the therapy. In one embodiment, the efficacy of a treatment of a patient for diabetic retinopathy is monitored by measuring one or more biomarkers selected from serpin family F member 1 (SERPINF1), retinoic acid receptor responder 2 (RARRES2), complement factor I (CFI), amyloid beta precursor protein (APP), complement C4A (C4A), and complement C4B (C4B); and evaluating the efficacy of the treatment, wherein detection of increased levels of the one or more biomarkers selected from SERPINF1, RARRES2, CFI, APP, C4A, and C4B in the second aqueous humor sample compared to the first aqueous humor sample indicate that the patient is worsening or not responding to the treatment, and detection of decreased levels of the one or more biomarkers selected from SERPINF1, RARRES2, CFI, APP, C4A, and C4B in the second aqueous humor sample compared to the first aqueous humor sample indicate that the patient is improving. In certain embodiments, the method further comprises altering the treatment if the patient is worsening or not responding to the treatment.

In another aspect, a method of diagnosing proliferative vitreoretinopathy (PVR) in a patient is provided, the method comprising: obtaining an aqueous humor sample from an eye of the patient; and measuring levels of one or more biomarkers selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein increased levels of the one or more biomarkers selected from IGFBP6, CCL15, CXCL12, VEGFA, and CCL23 compared to reference value ranges for the levels of the one or more biomarkers in a control aqueous humor sample indicate that the patient has PVR. The method may further comprise determining an appropriate treatment regimen for a patient and, in particular, whether a patient should be treated for PVR. For example, a patient is selected for treatment for PVR if the patient has a positive diagnosis for PVR based on a biomarker expression profile, as described herein. The treatment for PVR may comprise, for example, performing vitreous surgery, vitrectomy, membrane peeling, or retinotomy.

In some embodiments, the methods described herein are used for monitoring PVR in a subject. For example, a first aqueous humor sample can be obtained from the patient at a first time point and a second aqueous humor sample can be obtained from the subject at a second (later) time point. In one embodiment, PVR is monitored in the patient by measuring levels of one or more biomarkers in the first aqueous humor sample and the second aqueous humor sample, wherein the biomarkers are selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein detection of increased levels of the one or more biomarkers selected from IGFBP6, CCL15, CXCL12, VEGFA, and CCL23 in the second aqueous humor sample compared to the first aqueous humor sample indicate that the patient is worsening, and wherein detection of decreased levels of the one or more biomarkers selected from IGFBP6, CCL15, CXCL12, VEGFA, and CCL23 in the second aqueous humor sample compared to the first aqueous humor sample indicate that the patient is improving.

The subject methods may also be used for assaying pre-treatment and post-treatment aqueous humor samples obtained from an individual to determine whether the individual is responsive or not responsive to a treatment. For example, a first aqueous humor sample can be obtained from a subject before the subject undergoes the therapy, and a second aqueous humor sample can be obtained from the subject after the subject undergoes the therapy. In one embodiment, the efficacy of a treatment of a patient for PVR is monitored by measuring one or more biomarkers selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23); and evaluating the efficacy of the treatment, wherein detection of increased levels of the one or more biomarkers selected from IGFBP6, CCL15, CXCL12, VEGFA, and CCL23 in the second aqueous humor sample compared to the first aqueous humor sample indicate that the patient is worsening or not responding to the treatment, and detection of decreased levels of the one or more biomarkers selected from IGFBP6, CCL15, CXCL12, VEGFA, and CCL23 in the second aqueous humor sample compared to the first aqueous humor sample indicate that the patient is improving. In certain embodiments, the method further comprises altering the treatment if the patient is worsening or not responding to the treatment.

In another aspect, a method of diagnosing melanoma in a patient is provided, the method comprising: obtaining an aqueous humor sample from an eye of the patient; and measuring levels of one or more biomarkers selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein increased levels of the one or more biomarkers selected from FSTL1, ENPP2, ANG, MET, and HGF compared to reference value ranges for the levels of the one or more biomarkers in a control aqueous humor sample indicate that the patient has melanoma. The method may further comprise determining an appropriate treatment regimen for a patient and, in particular, whether a patient should be treated for melanoma. For example, a patient is selected for treatment for melanoma if the patient has a positive diagnosis for melanoma based on a biomarker expression profile, as described herein. The treatment for melanoma may comprise, for example, performing surgery to excise the melanoma. In some embodiments, the treatment further comprises performing radiation therapy or administering interferon, interleukin-2 (IL-2), dacarbazine, vemurafenib, dabrafenib, trametinib, pembrolizumab, ipilimumab, tremelimumab, nivolumab/relatlimab, or imiquimod, or a combination thereof.

In some embodiments, the methods described herein are used for monitoring melanoma in a subject. For example, a first aqueous humor sample can be obtained from the patient at a first time point and a second aqueous humor sample can be obtained from the subject at a second (later) time point. In one embodiment, melanoma is monitored in the patient by measuring levels of one or more biomarkers in the first aqueous humor sample and the second aqueous humor sample, wherein the biomarkers are selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein detection of increased levels of the one or more biomarkers selected from FSTL1, ENPP2, ANG, MET, and HGF in the second aqueous humor sample compared to the first aqueous humor sample indicate that the patient is worsening, and wherein detection of decreased levels of the one or more biomarkers selected from FSTL1, ENPP2, ANG, MET, and HGF in the second aqueous humor sample compared to the first aqueous humor sample indicate that the patient is improving.

The subject methods may also be used for assaying pre-treatment and post-treatment aqueous humor samples obtained from an individual to determine whether the individual is responsive or not responsive to a treatment. For example, a first aqueous humor sample can be obtained from a subject before the subject undergoes the therapy, and a second aqueous humor sample can be obtained from the subject after the subject undergoes the therapy. In one embodiment, the efficacy of a treatment of a patient for melanoma is monitored by measuring one or more biomarkers selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF); and evaluating the efficacy of the treatment, wherein detection of increased levels of the one or more biomarkers selected from FSTL1, ENPP2, ANG, MET, and HGF in the second aqueous humor sample compared to the first aqueous humor sample indicate that the patient is worsening or not responding to the treatment, and detection of decreased levels of the one or more biomarkers selected from FSTL1, ENPP2, ANG, MET, and HGF in the second aqueous humor sample compared to the first aqueous humor sample indicate that the patient is improving. In certain embodiments, the method further comprises altering the treatment if the patient is worsening or not responding to the treatment.

In another aspect, a method of diagnosing uveitis in a patient is provided, the method comprising: obtaining an aqueous humor sample from an eye of the patient; and measuring levels of one or more biomarkers selected from retinoic acid receptor responder 2 (RARRES2), biotinidase (BTD), cystatin C (CST3), TIMP metallopeptidase inhibitor 2 (TIMP2), and prostaglandin D2 synthase (PTGDS) in the aqueous humor sample, wherein increased levels of the one or more biomarkers selected from RARRES2, BTD, CST3, TIMP2, and PTGDS compared to reference value ranges for the levels of the one or more biomarkers in a control aqueous humor sample indicate that the patient has uveitis. The method may further comprise determining an appropriate treatment regimen for a patient and, in particular, whether a patient should be treated for uveitis. For example, a patient is selected for treatment for uveitis if the patient has a positive diagnosis for uveitis based on a biomarker expression profile, as described herein. The treatment for non-infectious uveitis may comprise, for example, administering a therapeutically effective amount of one or more anti-inflammatory or immunosuppressive agents such as, but not limited to, glucocorticoid steroids including, without limitation, prednisolone, methylprednisolone, iluvien, ozurdex, retisert, and triamcinolone; T-cell inhibitors including, without limitation, calcineurin inhibitors such as cyclosporine, tacrolimus and voclosporin, and mTOR inhibitors such as everolimus and sirolimus; antimetabolites including, without limitation, purine antagonists such as azathioprine, dihydrofolate reductase (DHFR) inhibitors such as methotrexate, and inosine monophosphate dehydrogenase (IMPDH) inhibitors such as mycophenolate mofetil; anti-TNF agents including, without limitation, adalimumab, certolizumab, golimumab, infliximab, and etanercept; biologic agents including, without limitation, efalizumab, rituximab, abatacept, alemtuzumab, anakinra, canakinumab, gevokizumab, daclizumab, tocilizumab, secukinumab, interferon α/β, fingolimod, aflibercept, bevacizumab, ranibizumab, and intravenous immunoglobulin (IVIG); alkylating agents including, without limitation, chlorambucil and cyclophosphamide; and cycloplegic agents including, without limitation, atropine and homatropine; or a combination thereof. The treatment for bacterial uveitis may comprise, for example, administering a therapeutically effective amount of one or more antibiotics such as, but not limited to, vancomycin, ceftazidime, amikacin, gentamycin, moxifloxacin, and cephalosporins such as cefacetrile (cephacetrile), cefadroxil (cefadroxyl; Duricef), cefalexin (cephalexin; Keflex), cefaloglycin (cephaloglycin), cefalonium (cephalonium), cefaloridine (cephaloradine), cefalotin (cephalothin; Keflin), cefapirin (cephapirin; Cefadryl), cefatrizine, cefazaflur, cefazedone, cefazolin (cephazolin; Ancef, Kefzol), cefradine (cephradine; Velosef), cefroxadine, ceftezole, cefaclor (Ceclor, Distaclor, Keflor, Raniclor), cefonicid (Monocid), cefprozil (cefproxil; Cefzil), cefuroxime (Zefu, Zinnat, Zinacef, Ceftin, Biofuroksym, Xorimax), cefuzonam, cefmetazole, cefotetan, cefoxitin, loracarbef (Lorabid), cefbuperazone, cefmetazole (Zefazone), cefminox, cefotetan (Cefotan), cefoxitin (Mefoxin), cefotiam (Pansporin), cefcapene, cefdaloxime, cefdinir (Sefdin, Zinir, Omnicef, Kefnir), cefditoren, cefetamet, cefixime (Fixx, Zifi, Suprax), cefmenoxime, cefodizime, cefotaxime (Claforan), cefovecin (Convenia), cefpimizole, cefpodoxime (Vantin, PECEF, Simplicef), cefteram, ceftibuten (Cedax), ceftiofur (Naxcel, Excenel), ceftiolene, ceftizoxime (Cefizox), ceftriaxone (Rocephin), cefoperazone (Cefobid), ceftazidime (Meezat, Fortum, Fortaz), latamoxef (moxalactam), cefclidine, cefepime (Maxipime), cefluprenam, cefoselis, cefozopran, cefpirome (Cefrom), cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; or a combination thereof. The treatment for viral uveitis may comprise, for example, administering a therapeutically effective amount of one or more antiviral agents such as, but not limited to, ganciclovir, acyclovir, foscarnet, valacyclovir, and cidofivir, or a combination thereof. The treatment for fungal uveitis may comprise, for example, administering a therapeutically effective amount of one or more antifungal agents such as, but not limited to, amphotericin B, voriconazole, caspofungin, and fluconazole, or a combination thereof.

In some embodiments, the methods described herein are used for monitoring uveitis in a subject. For example, a first aqueous humor sample can be obtained from the patient at a first time point and a second aqueous humor sample can be obtained from the subject at a second (later) time point. In one embodiment, uveitis is monitored in the patient by measuring levels of one or more biomarkers in the first aqueous humor sample and the second aqueous humor sample, wherein the biomarkers are selected from RARRES2, BTD, CST3, TIMP2, and PTGDS in the aqueous humor sample, wherein detection of increased levels of the one or more biomarkers selected from RARRES2, BTD, CST3, TIMP2, and PTGDS in the second aqueous humor sample compared to the first aqueous humor sample indicate that the patient is worsening, and wherein detection of decreased levels of the one or more biomarkers selected from RARRES2, BTD, CST3, TIMP2, and PTGDS in the second aqueous humor sample compared to the first aqueous humor sample indicate that the patient is improving.

The subject methods may also be used for assaying pre-treatment and post-treatment aqueous humor samples obtained from an individual to determine whether the individual is responsive or not responsive to a treatment. For example, a first aqueous humor sample can be obtained from a subject before the subject undergoes the therapy, and a second aqueous humor sample can be obtained from the subject after the subject undergoes the therapy. In one embodiment, the efficacy of a treatment of a patient for uveitis is monitored by measuring one or more biomarkers selected from RARRES2, BTD, CST3, TIMP2, and PTGDS; and evaluating the efficacy of the treatment, wherein detection of increased levels of the one or more biomarkers selected from RARRES2, BTD, CST3, TIMP2, and PTGDS in the second aqueous humor sample compared to the first aqueous humor sample indicate that the patient is worsening or not responding to the treatment, and detection of decreased levels of the one or more biomarkers selected from RARRES2, BTD, CST3, TIMP2, and PTGDS in the second aqueous humor sample compared to the first aqueous humor sample indicate that the patient is improving. In certain embodiments, the method further comprises altering the treatment if the patient is worsening or not responding to the treatment.

In another aspect, a method of diagnosing neovascular inflammatory vitreoretinopathy (NIV) in a patient is provided, the method comprising: obtaining an aqueous humor sample from an eye of the patient; measuring levels of one or more biomarkers selected from serpin family C member 1 (SERPINC1), hemopexin (HPX), coagulation factor II (F2), complement C9 (C9), and complement C6 (C6) in the aqueous humor sample, wherein increased levels of the one or more biomarkers selected from SERPINC1, HPX, F2, C9, and C6 compared to reference value ranges for the levels of the one or more biomarkers in a control aqueous humor sample indicate that the patient has NIV. The method may further comprise determining an appropriate treatment regimen for a patient and, in particular, whether a patient should be treated for NIV. For example, a patient is selected for treatment for NIV if the patient has a positive diagnosis for NIV based on a biomarker expression profile, as described herein. The treatment for NIV may comprise, for example, administering a fluocinolone acetonide, dexamethasone, or bevacizumab, or performing panretinal scatter photocoagulation (PRP), vitrectomy, trabeculectomy, or a combination thereof.

In some embodiments, the methods described herein are used for monitoring NIV in a subject. For example, a first aqueous humor sample can be obtained from the patient at a first time point and a second aqueous humor sample can be obtained from the subject at a second (later) time point. In one embodiment, NIV is monitored in the patient by measuring levels of one or more biomarkers in the first aqueous humor sample and the second aqueous humor sample, wherein the biomarkers are selected from serpin family C member 1 (SERPINC1), hemopexin (HPX), coagulation factor II (F2), complement C9 (C9), and complement C6 (C6) in the aqueous humor sample, wherein detection of increased levels of the one or more biomarkers selected from SERPINC1, HPX, F2, C9, and C6 in the second aqueous humor sample compared to the first aqueous humor sample indicate that the patient is worsening, and wherein detection of decreased levels of the one or more biomarkers selected from SERPINC1, HPX, F2, C9, and C6 in the second aqueous humor sample compared to the first aqueous humor sample indicate that the patient is improving.

The subject methods may also be used for assaying pre-treatment and post-treatment aqueous humor samples obtained from an individual to determine whether the individual is responsive or not responsive to a treatment. For example, a first aqueous humor sample can be obtained from a subject before the subject undergoes the therapy, and a second aqueous humor sample can be obtained from the subject after the subject undergoes the therapy. In one embodiment, the efficacy of a treatment of a patient for NIV is monitored by measuring one or more biomarkers selected from serpin family C member 1 (SERPINC1), hemopexin (HPX), coagulation factor II (F2), complement C9 (C9), and complement C6 (C6); and evaluating the efficacy of the treatment, wherein detection of increased levels of the one or more biomarkers selected from SERPINC1, HPX, F2, C9, and C6 in the second aqueous humor sample compared to the first aqueous humor sample indicate that the patient is worsening or not responding to the treatment, and detection of decreased levels of the one or more biomarkers selected from SERPINC1, HPX, F2, C9, and C6 in the second aqueous humor sample compared to the first aqueous humor sample indicate that the patient is improving. In certain embodiments, the method further comprises altering the treatment if the patient is worsening or not responding to the treatment.

In some cases, the diagnostic methods described herein may be used by themselves or combined with medical imaging or other ophthalmology techniques for detecting ocular lesions to confirm the diagnosis and further evaluate the severity and extent of disease (e.g., detect retina damage, leaking, macular degeneration, inflammation, neovascularization, or other abnormalities of retinal arteries and veins, and/or macular edema) to aid in determining prognosis and evaluating optimal strategies for treatment (e.g., laser surgery, laser coagulation, vitrectomy, or injection of corticosteroids (e.g., triamcinolone) or vascular endothelial growth factor inhibitors (e.g., ranibizumab) into the eye, etc.). Exemplary medical imaging and ophthalmology techniques include, without limitation, fundus photography, fluorescein angiography, retinal vessel analysis, ultrasonography, optical coherence tomography, autofluorescence, indocyanine green angiography, and the radioactive phosphorus uptake test on the eye.

In some cases, combinations of biomarkers or combinations of biomarker panels are used in the subject methods. In some such cases, the levels of all measured biomarkers must change (as described above) in order for the diagnosis to be made. In some embodiments, only some biomarkers are used in the methods described herein. For example, a single biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, or 20 biomarkers can be used in any combination. In other embodiments, all the biomarkers are used. The quantitative values may be combined in linear or non-linear fashion to calculate one or more risk scores for a disease (e.g., age-related macular degeneration, diabetic retinopathy, proliferative vitreoretinopathy, uveitis, neovascular inflammatory vitreoretinopathy, or melanoma) or the biological age for an individual and risk of age-related pathology).

The level of a biomarker in a pre-treatment aqueous humor sample can be referred to as a “pre-treatment value” because the first aqueous humor sample is isolated from the individual prior to the administration of the therapy (i.e., “pre-treatment”). The level of a biomarker in the pre-treatment aqueous humor sample can also be referred to as a “baseline value” because this value is the value to which “post-treatment” values are compared. In some cases, the baseline value (i.e., “pre-treatment value”) is determined by determining the level of a biomarker in multiple (i.e., more than one, e.g., two or more, three or more, for or more, five or more, etc.) pre-treatment aqueous humor samples. In some cases, the multiple pre-treatment aqueous humor samples are isolated from an individual at different time points in order to assess natural fluctuations in biomarker levels prior to treatment. As such, in some cases, one or more (e.g., two or more, three or more, for or more, five or more, etc.) pre-treatment aqueous humor samples are isolated from the individual. In some embodiments, all of the pre-treatment aqueous humor samples will be the same type of aqueous humor sample (e.g., a biopsy sample). In some cases, two or more pre-treatment aqueous humor samples are pooled prior to determining the level of the biomarker in the aqueous humor samples. In some cases, the level of the biomarker is determined separately for two or more pre-treatment aqueous humor samples and a “pre-treatment value” is calculated by averaging the separate measurements.

A post-treatment aqueous humor sample is isolated from an individual after the administration of a therapy. Thus, the level of a biomarker in a post-treatment sample can be referred to as a “post-treatment value”. In some embodiments, the level of a biomarker is measured in additional post-treatment aqueous humor samples (e.g., a second, third, fourth, fifth, etc. post-treatment aqueous humor sample). Because additional post-treatment aqueous humor samples are isolated from the individual after the administration of a treatment, the levels of a biomarker in the additional aqueous humor samples can also be referred to as “post-treatment values.”

The term “responsive” as used herein means that the treatment is having the desired effect such as improving vision, preventing, reducing or delaying vision loss, preventing or reducing retina damage, preventing or reducing neovascularization, and/or preventing or reducing macular edema. When the individual does not improve in response to the treatment, it may be desirable to seek a different therapy or treatment regime for the individual.

The determination that an individual has a certain biological age, is at risk of age-related pathology and morbidity, or has a disease by expression profiling is an active clinical application of the correlation between levels of a biomarker and biological age, risk of age-related pathology and morbidity, or a certain disease. For example, “determining” requires the active step of reviewing the data, which is produced during the active assaying step(s), and determining biological age or resolving whether an individual does or does not have a disease or risk of a disease. Additionally, in some cases, a decision is made to proceed with a current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in treating proliferative diabetic retinopathy, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated for proliferative diabetic retinopathy by administering a vascular endothelial growth factor inhibitor. Then to “continue treatment” would be to continue with this type of treatment. If the current course of treatment is not effective, the treatment may be altered, e.g., switching treatment to a different vascular endothelial growth factor inhibitor or increasing the dose or frequency of administration of the vascular endothelial growth factor inhibitor, or changing to a different type of treatment such as laser surgery, laser coagulation, or vitrectomy.

In other words, the level of one or more biomarkers may be monitored in order to determine when to continue therapy and/or when to alter therapy. As such, a post-treatment aqueous humor sample can be isolated after any of the administrations and the aqueous humor sample can be assayed to determine the level of a biomarker. Accordingly, the subject methods can be used to determine whether an individual being treated for proliferative diabetic retinopathy is responsive or is maintaining responsiveness to a treatment.

The therapy can be administered to an individual any time after a pre-treatment aqueous humor sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment aqueous humor sample is isolated (or, when multiple pre-treatment aqueous humor samples are isolated, after the final pre-treatment aqueous humor sample is isolated).

In some cases, more than one type of therapy may be administered to the individual. For example, a subject who has proliferative diabetic retinopathy may be treated with a corticosteroid and a vascular endothelial growth factor inhibitor or laser surgery. A subject who has more severe disease or who is at high risk of disease progression, may be treated more aggressively. For example, treatment of a high-risk patient may include, without limitation, laser surgery, laser coagulation, or vitrectomy.

In some embodiments, the subject methods include providing an analysis (e.g., an oral or written report) having any or all of the following information: identifying information of the subject (name, age, etc.), a description of what type of aqueous humor sample(s) was used and/or how it was used, the technique used to assay the sample, the results of the assay (e.g., the level of the biomarker as measured, and/or the fold-change of a biomarker level over time, or in a post-treatment assay compared to a pre-treatment assay), the assessment as to whether the individual is determined to have melanoma or a vitreoretinal disease, the predicted biological age of the individual and risk of age-related pathology and morbidity, a recommendation for additional screening for pathology, a recommendation for treatment, and/or to continue or alter therapy, a recommended strategy for additional therapy, etc. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites or metabolic byproducts) that are indicative of the expression level of the biomarker. The methods for measuring biomarkers in a sample have many applications. For example, one or more biomarkers can be measured to aid in diagnosing a patient with a disease or risk of age-related pathology and determining the appropriate treatment for a subject, as well as monitoring responses of a subject to treatment.

In some embodiments, the amount or level in the sample of one or more proteins/polypeptides encoded by a gene of interest is determined. Any convenient protocol for evaluating protein levels may be employed, wherein the level of one or more proteins in the assayed sample is determined. Two representative and convenient techniques for assaying protein levels include aptamer-based assays and antibody-based methods such as the enzyme-linked immunosorbent assay (ELISA).

Aptamer-based assays use aptamers comprising single-stranded oligonucleotides that bind specifically to biomarker proteins of interest. Either high affinity RNA or DNA aptamers with specificity for a protein of interest may be used. Functional groups that mimic amino acid side-chains may be added to aptamers to confer protein-like properties to improve binding affinity to a protein of interest. Aptamers that bind specifically and with high affinity to a protein of interest can be selected from large libraries of aptamers having randomized sequences using Systematic Evolution of Ligands by EXponential enrichment (SELEX). The aptamers may be designed with unique nucleotide sequences recognizable by specific hybridization probes for capture on a hybridization array for multiplexed detection of biomarkers (see, e.g., Gold et al. (2010) Aptamer-Based Multiplexed Proteomic Technology for Biomarker Discovery. PLoS ONE 5(12):e15004; herein incorporated by reference in its entirety.

For antibody-based methods of protein level determination, any convenient antibody can be used that specifically binds to the intended biomarker. The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g., urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, other methods for measuring the levels of one or more proteins in a sample may be employed, and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; mass spectrometry, or tandem mass spectrometry, proteomic arrays, xMAP microsphere technology, western blotting, immunohistochemistry, flow cytometry, cytometry by time-of-flight (CyTOF), multiplexed ion beam imaging (MIBI), and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

For measuring protein activity levels, the amount or level of protein activity in the sample of one or more proteins/polypeptides encoded by the gene of interest is determined.

In other embodiments, the amount or level in the sample of one or more proteins is determined. Any convenient method for measuring protein levels in a sample may be used, e.g., antibody-based methods, e.g., aptamer-based assays, immunoassay such as enzyme-linked immunosorbent assays (ELISAs), immunohistochemistry, and mass spectrometry.

The resultant data provides information regarding expression, amount, and/or activity for each of the biomarkers that have been measured, wherein the information is in terms of whether or not the biomarker is present (e.g., expressed) and at what level, and wherein the data may be both qualitative and quantitative.

### Data Analysis

In some embodiments, one or more pattern recognition methods can be used in analyzing the data for biomarker levels. The quantitative values may be combined in linear or non-linear fashion to calculate one or more risk scores for a disease (e.g., age-related macular degeneration, diabetic retinopathy, proliferative vitreoretinopathy, uveitis, neovascular inflammatory vitreoretinopathy, or melanoma) or the biological age for an individual and risk of age-related pathology. In some embodiments, measurements for a biomarker or combinations of biomarkers are formulated into linear or non-linear models or algorithms (e.g., a ‘biomarker signature’) and converted into a likelihood score. This likelihood score indicates the probability that an aqueous humor sample is from a patient of a particular biological age, or a patient who may exhibit no evidence of disease, or a patient who may exhibit a particular disease or a risk of age-related pathology and morbidity. The models and/or algorithms can be provided in machine readable format, and may be used to correlate biomarker levels or a biomarker profile with a disease state, and/or to designate a treatment modality for a patient or class of patients.

Analyzing the levels of a plurality of biomarkers may comprise the use of an algorithm or classifier. In some embodiments, a machine learning algorithm is used to classify a patient as having proliferative diabetic retinopathy. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Kits

Also provided are kits for use in the methods, disclosed herein, for predicting biological age, determining risk of age-related pathology and morbidity, and/or diagnosing a disease (e.g., age-related macular degeneration, diabetic retinopathy, proliferative vitreoretinopathy, uveitis, neovascular inflammatory vitreoretinopathy, or melanoma). The subject kits include agents (e.g., an aptamer or antibody that specifically binds to a biomarker and/or other assay reagents, and the like) for determining the level of at least one biomarker. In some embodiments, a kit comprises agents for determining the level of a single biomarker, two or more different biomarkers, three or more different biomarkers, four or more different biomarkers, or five or more different biomarkers. In certain embodiments, the kit comprises reagents for performing an aptamer-based assay or immunoassay.

In certain embodiments, a kit for predicting biological age and/or determining risk of age-related pathology and morbidity is provided, the kit comprising agents for detecting at least 3 biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1). In some embodiments, the kit comprises agents for detecting all of the MMP10, NRP1, SEMA3C, HES5, and FGFRL1 biomarkers. In some embodiments, the kit comprises an aptamer or antibody that specifically binds to MMP10, an aptamer or antibody that specifically binds to NRP1, an aptamer or antibody that specifically binds to SEMA3C, an aptamer or antibody that specifically binds to HES5, and an aptamer or antibody that specifically binds to FGFRL1.

In another aspect, a kit for diagnosing age-related macular degeneration (AMD) is provided, the kit comprising agents for detecting at least 3 biomarkers selected from the group consisting of serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD). In certain embodiments, the kit comprises agents for detecting all of the SERPINF1, VEGFA, FLT1, VEGFB, and VEGFD biomarkers. In some embodiments, the kit comprises an aptamer or antibody that specifically binds to SERPINF1, an aptamer or antibody that specifically binds to VEGFA, an aptamer or antibody that specifically binds to FLT1, an aptamer or antibody that specifically binds to VEGFB, and an aptamer or antibody that specifically binds to VEGFD.

In another aspect, a kit for diagnosing diabetic retinopathy is provided, the kit comprising agents for detecting at least 3 biomarkers selected from the group consisting of serpin family F member 1 (SERPINF1), retinoic acid receptor responder 2 (RARRES2), complement factor I (CFI), amyloid beta precursor protein (APP), complement C4A (C4A), and complement C4B (C4B). In certain embodiments, the kit comprises agents for detecting all of the SERPINF1, RARRES2, CFI, APP, C4A, and C4B biomarkers. In certain embodiments, the kit comprises an aptamer or antibody that specifically binds to SERPINF1, an aptamer or antibody that specifically binds to RARRES2, an aptamer or antibody that specifically binds to CFI, an aptamer or antibody that specifically binds to APP, an aptamer or antibody that specifically binds to C4A, and an aptamer or antibody that specifically binds to C4B.

In another aspect, a kit for diagnosing proliferative vitreoretinopathy (PVR) is provided, the kit comprising agents for detecting at least 3 biomarkers selected from the group consisting of insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23). In certain embodiments, the kit comprises agents for detecting all of the IGFBP6, CCL15, CXCL12, VEGFA, and CCL23 biomarkers. In some embodiments, the kit comprises an aptamer or antibody that specifically binds to IGFBP6, an aptamer or antibody that specifically binds to CCL15, an aptamer or antibody that specifically binds to CXCL12, an aptamer or antibody that specifically binds to VEGFA, and an aptamer or antibody that specifically binds to CCL23.

In another aspect, a kit for diagnosing melanoma is provided, the kit comprising agents for detecting at least 3 biomarkers selected from the group consisting of follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF). In certain embodiments, the kit comprises agents for detecting all of the FSTL1, ENPP2, ANG, MET, and HGF biomarkers. In some embodiments, the kit comprises an aptamer or antibody that specifically binds to FSTL1, an aptamer or antibody that specifically binds to ENPP2, an aptamer or antibody that specifically binds to ANG, an aptamer or antibody that specifically binds to MET, and an aptamer or antibody that specifically binds to HGF.

In another aspect, a kit for diagnosing uveitis is provided, the kit comprising agents for detecting at least 3 biomarkers selected from the group consisting of retinoic acid receptor responder 2 (RARRES2), biotinidase (BTD), cystatin C (CST3), TIMP metallopeptidase inhibitor 2 (TIMP2), and prostaglandin D2 synthase (PTGDS). In certain embodiments, the kit comprises agents for detecting all of the RARRES2, BTD, CST3, TIMP2, and PTGDS biomarkers. In some embodiments, the kit comprises an aptamer or antibody that specifically binds to RARRES2, an aptamer or antibody that specifically binds to BTD, an aptamer or antibody that specifically binds to CST3, an aptamer or antibody that specifically binds to TIMP2, and an aptamer or antibody that specifically binds to PTGDS.

In another aspect, a kit for diagnosing neovascular inflammatory vitreoretinopathy (NIV) is provided, the kit comprising agents for detecting at least 3 biomarkers selected from the group consisting of serpin family C member 1 (SERPINC1), hemopexin (HPX), coagulation factor II (F2), complement C9 (C9), and complement C6 (C6). In certain embodiments, the kit comprises agents for detecting all of the SERPINC1, HPX, F2, C9, and C6 biomarkers. In some embodiments, the kit comprises an aptamer or antibody that specifically binds to SERPINC1, an aptamer or antibody that specifically binds to HPX, an aptamer or antibody that specifically binds to F2, an aptamer or antibody that specifically binds to C9, and an aptamer or antibody that specifically binds to C6.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-128 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of diagnosing and treating age-related macular degeneration (AMD) in a patient, the method comprising:
obtaining an aqueous humor sample from an eye of the patient;
measuring levels of one or more biomarkers selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) in the aqueous humor sample, wherein increased levels of the one or more biomarkers selected from SERPINF1, VEGFA, FLT1, VEGFB, and VEGFD compared to reference value ranges for the levels of the one or more biomarkers in a control aqueous humor sample indicate that the patient has AMD; and
treating the patient for the AMD if the patient has a positive diagnosis for AMD.

2. The method of aspect 1, wherein the levels of at least two, at least three, or at least four biomarkers selected from SERPINF1, VEGFA, FLT1, VEGFB, and VEGFD are measured in the aqueous humor sample.

3. The method of aspect 2, wherein the levels of SERPINF1, VEGFA, FLT1, VEGFB, and VEGFD are measured in the aqueous humor sample.

4. The method of any one of aspects 1-3, wherein said treating the patient for the AMD comprises administering vitamin C, zinc, vitamin E, copper, beta-carotene, lutein, zeaxanthin, ranibizumab, aflibercept, brolucizumab, or faricimab, or a combination thereof to the patient.

5. The method of any one of aspects 1-4, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.

6. The method of aspect 5, wherein the aptamer-based proteomic assay is performed using a multiplex aptamer array.

7. The method of any one of aspects 1-7, wherein the subject has not yet developed clinical symptoms.

8. The method of any one of aspects 1-7, wherein the subject has developed clinical symptoms.

9. A method of monitoring age-related macular degeneration (AMD) in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) in the aqueous humor sample, wherein detection of increased levels of the one or more biomarkers selected from SERPINF1, VEGFA, FLT1, VEGFB, and VEGFD in the second aqueous humor sample compared to the first aqueous humor sample indicate that the patient is worsening, and wherein detection of decreased levels of the one or more biomarkers selected from SERPINF1, VEGFA, FLT1, VEGFB, and VEGFD in the second aqueous humor sample compared to the first aqueous humor sample indicate that the patient is improving.

10. A method of monitoring efficacy of a treatment of a patient for age-related macular degeneration (AMD), the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD); and
evaluating the efficacy of the treatment, wherein detection of increased levels of the one or more biomarkers selected from SERPINF1, VEGFA, FLT1, VEGFB, and VEGFD in the second aqueous humor sample compared to the first aqueous humor sample indicate that the patient is worsening or not responding to the treatment, and detection of decreased levels of the one or more biomarkers selected from SERPINF1, VEGFA, FLT1, VEGFB, and VEGFD in the second aqueous humor sample compared to the first aqueous humor sample indicate that the patient is improving.

11. The method of aspect 10, further comprising altering the treatment if the patient is worsening or not responding to the treatment.

12. A kit for diagnosing age-related macular degeneration (AMD) comprising agents for detecting at least 3 biomarkers selected from the group consisting of serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD).

13. The kit of aspect 12, wherein the kit comprises agents for detecting all of the SERPINF1, VEGFA, FLT1, VEGFB, and VEGFD biomarkers.

14. The kit of aspect 12 or 13, further comprising reagents for performing an aptamer-based proteomic assay or immunoassay.

15. The kit of aspect 14, wherein the kit comprises an aptamer or antibody that specifically binds to SERPINF1, an aptamer or antibody that specifically binds to VEGFA, an aptamer or antibody that specifically binds to FLT1, an aptamer or antibody that specifically binds to VEGFB, and an aptamer or antibody that specifically binds to VEGFD.

16. The kit of any one of aspects 12-15, further comprising instructions for determining whether a subject has AMD.

17. A protein selected from the group consisting of serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) for use as a biomarker in diagnosing age-related macular degeneration (AMD).

18. An in vitro method of diagnosing macular degeneration (AMD), the method comprising:
obtaining an aqueous humor sample from an eye of the patient; and
measuring levels of one or more biomarkers selected from serpin family F member 1 (SERPINF1), vascular endothelial growth factor A (VEGFA), fms related receptor tyrosine kinase 1 (FLT1), vascular endothelial growth factor B (VEGFB), and vascular endothelial growth factor D (VEGFD) in the aqueous humor sample, wherein increased levels of the one or more biomarkers selected from SERPINF1, VEGFA, FLT1, VEGFB, and VEGFD compared to reference value ranges for the levels of the one or more biomarkers in a control aqueous humor sample indicate that the patient has AMD.

19. A method of diagnosing and treating diabetic retinopathy in a patient, the method comprising:
obtaining an aqueous humor sample from an eye of the patient;
measuring levels of one or more biomarkers selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein increased levels of the one or more biomarkers selected from SERPINF1, RARRES2, CFI, APP, C4A, and C4B compared to reference value ranges for the levels of the one or more biomarkers in a control aqueous humor sample indicate that the patient has diabetic retinopathy; and
treating the patient for the diabetic retinopathy if the patient has a positive diagnosis for diabetic retinopathy.

20. The method of aspect 19, wherein the levels of at least two, at least three, or at least four biomarkers selected from SERPINF1, RARRES2, CFI, APP, C4A, and C4B are measured in the aqueous humor sample.

21. The method of aspect 20, wherein the levels of SERPINF1, RARRES2, CFI, APP, C4A, and C4B are measured in the aqueous humor sample.

22. The method of any one of aspects 19-21, wherein said treating the patient for the diabetic retinopathy comprises administering an anti-vascular endothelial growth factor (VEGF) agent or a steroid, or performing panretinal laser photocoagulation or a vitrectomy, or a combination thereof.

23. The method of aspect 22, wherein the anti-VEGF agent is bevacizumab, ranibizumab, sunitinib, sorafenib, axitinib, aflibercept, brolucizuma, faricimab, or pazopanib.

24. The method of aspect 22, wherein the steroid is triamcinolone acetonide, fluocinolone acetonide, or dexamethasone.

25. The method of any one of aspects 19-24, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.

26. The method of aspect 25, wherein the aptamer-based proteomic assay is performed using a multiplex aptamer array.

27. The method of any one of aspects 19-26, wherein the subject has not yet developed clinical symptoms.

28. The method of any one of aspects 19-26, wherein the subject has developed clinical symptoms.

29. A method of monitoring diabetic retinopathy in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein detection of increased levels of the one or more biomarkers selected from SERPINF1, RARRES2, CFI, APP, C4A, and C4B in the second aqueous humor sample compared to the first aqueous humor sample indicate that the patient is worsening, and wherein detection of decreased levels of the one or more biomarkers selected from SERPINF1, RARRES2, CFI, APP, C4A, and C4B in the second aqueous humor sample compared to the first aqueous humor sample indicate that the patient is improving.

30. A method of monitoring efficacy of a treatment of a patient for diabetic retinopathy, the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from serpin family F member 1 (SERPINF1), retinoic acid receptor responder 2 (RARRES2), complement factor I (CFI), amyloid beta precursor protein (APP), complement C4A (C4A), and complement C4B (C4B); and
evaluating the efficacy of the treatment, wherein detection of increased levels of the one or more biomarkers selected from SERPINF1, RARRES2, CFI, APP, C4A, and C4B in the second aqueous humor sample compared to the first aqueous humor sample indicate that the patient is worsening or not responding to the treatment, and detection of decreased levels of the one or more biomarkers selected from SERPINF1, RARRES2, CFI, APP, C4A, and C4B in the second aqueous humor sample compared to the first aqueous humor sample indicate that the patient is improving.

31. The method of aspect 30, further comprising altering the treatment if the patient is worsening or not responding to the treatment.

32. A kit for diagnosing diabetic retinopathy comprising agents for detecting at least 3 biomarkers selected from the group consisting of serpin family F member 1 (SERPINF1), retinoic acid receptor responder 2 (RARRES2), complement factor I (CFI), amyloid beta precursor protein (APP), complement C4A (C4A), and complement C4B (C4B).

33. The kit of aspect 32, wherein the kit comprises agents for detecting all of the SERPINF1, RARRES2, CFI, APP, C4A, and C4B biomarkers.

34. The kit of aspect 32 or 33, further comprising reagents for performing an aptamer-based proteomic assay or immunoassay.

35. The kit of aspect 34, wherein the kit comprises an aptamer or antibody that specifically binds to SERPINF1, an aptamer or antibody that specifically binds to RARRES2, an aptamer or antibody that specifically binds to CFI, an aptamer or antibody that specifically binds to APP, an aptamer or antibody that specifically binds to C4A, and an aptamer or antibody that specifically binds to C4B.

36. The kit of any one of aspects 32-35, further comprising instructions for determining whether a subject has diabetic retinopathy.

37. A protein selected from the group consisting of serpin family F member 1 (SERPINF1), retinoic acid receptor responder 2 (RARRES2), complement factor I (CFI), amyloid beta precursor protein (APP), complement C4A (C4A), and complement C4B (C4B) for use as a biomarker in diagnosing diabetic retinopathy.

38. An in vitro method of diagnosing diabetic retinopathy, the method comprising:
obtaining an aqueous humor sample from an eye of the patient; and
measuring levels of one or more biomarkers selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein increased levels of the one or more biomarkers selected from SERPINF1, RARRES2, CFI, APP, C4A, and C4B compared to reference value ranges for the levels of the one or more biomarkers in a control aqueous humor sample indicate that the patient has diabetic retinopathy.

39. A method of diagnosing and treating proliferative vitreoretinopathy (PVR) in a patient, the method comprising:
obtaining an aqueous humor sample from an eye of the patient;
measuring levels of one or more biomarkers selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein increased levels of the one or more biomarkers selected from IGFBP6, CCL15, CXCL12, VEGFA, and CCL23 compared to reference value ranges for the levels of the one or more biomarkers in a control aqueous humor sample indicate that the patient has PVR; and
treating the patient for the PVR if the patient has a positive diagnosis for PVR.

40. The method of aspect 39, wherein the levels of at least two, at least three, or at least four biomarkers selected from IGFBP6, CCL15, CXCL12, VEGFA, and CCL23 are measured in the aqueous humor sample.

41. The method of aspect 40, wherein the levels of IGFBP6, CCL15, CXCL12, VEGFA, and CCL23 are measured in the aqueous humor sample.

42. The method of any one of aspects 39-41, wherein said treating the patient for the PVR comprises performing vitreous surgery, vitrectomy, membrane peeling, or retinotomy.

43. The method of any one of aspects 39-42, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.

44. The method of aspect 43, wherein the aptamer-based proteomic assay is performed using a multiplex aptamer array.

45. The method of any one of aspects 39-44, wherein the subject has not yet developed clinical symptoms.

46. The method of any one of aspects 39-44, wherein the subject has developed clinical symptoms.

47. A method of monitoring proliferative vitreoretinopathy (PVR) in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein detection of increased levels of the one or more biomarkers selected from IGFBP6, CCL15, CXCL12, VEGFA, and CCL23 in the second aqueous humor sample compared to the first aqueous humor sample indicate that the patient is worsening, and wherein detection of decreased levels of the one or more biomarkers selected from IGFBP6, CCL15, CXCL12, VEGFA, and CCL23 in the second aqueous humor sample compared to the first aqueous humor sample indicate that the patient is improving.

48. A method of monitoring efficacy of a treatment of a patient for proliferative vitreoretinopathy (PVR), the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23); and
evaluating the efficacy of the treatment, wherein detection of increased levels of the one or more biomarkers selected from IGFBP6, CCL15, CXCL12, VEGFA, and CCL23 in the second aqueous humor sample compared to the first aqueous humor sample indicate that the patient is worsening or not responding to the treatment, and detection of decreased levels of the one or more biomarkers selected from IGFBP6, CCL15, CXCL12, VEGFA, and CCL23 in the second aqueous humor sample compared to the first aqueous humor sample indicate that the patient is improving.

49. The method of aspect 48, further comprising altering the treatment if the patient is worsening or not responding to the treatment.

50. A kit for diagnosing proliferative vitreoretinopathy (PVR) comprising agents for detecting at least 3 biomarkers selected from the group consisting of insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23).

51. The kit of aspect 50, wherein the kit comprises agents for detecting all of the IGFBP6, CCL15, CXCL12, VEGFA, and CCL23 biomarkers.

52. The kit of aspect 50 or 51, further comprising reagents for performing an aptamer-based proteomic assay or immunoassay.

53. The kit of aspect 52, wherein the kit comprises an aptamer or antibody that specifically binds to IGFBP6, an aptamer or antibody that specifically binds to CCL15, an aptamer or antibody that specifically binds to CXCL12, an aptamer or antibody that specifically binds to VEGFA, and an aptamer or antibody that specifically binds to CCL23.

54. The kit of any one of aspects 50-53, further comprising instructions for determining whether a subject has PVR.

55. A protein selected from the group consisting of insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) for use as a biomarker in diagnosing proliferative vitreoretinopathy (PVR).

56. An in vitro method of diagnosing proliferative vitreoretinopathy (PVR), the method comprising:
obtaining an aqueous humor sample from an eye of the patient; and
measuring levels of one or more biomarkers selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein increased levels of the one or more biomarkers selected from IGFBP6, CCL15, CXCL12, VEGFA, and CCL23 compared to reference value ranges for the levels of the one or more biomarkers in a control aqueous humor sample indicate that the patient has PVR.

57. A method of diagnosing and treating melanoma in a patient, the method comprising:
obtaining an aqueous humor sample from an eye of the patient;
measuring levels of one or more biomarkers selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein increased levels of the one or more biomarkers selected from FSTL1, ENPP2, ANG, MET, and HGF compared to reference value ranges for the levels of the one or more biomarkers in a control aqueous humor sample indicate that the patient has melanoma; and
treating the patient for the melanoma if the patient has a positive diagnosis for melanoma.

58. The method of aspect 57, wherein the levels of at least two, at least three, or at least four biomarkers selected from FSTL1, ENPP2, ANG, MET, and HGF are measured in the aqueous humor sample.

59. The method of aspect 58, wherein the levels of FSTL1, ENPP2, ANG, MET, and HGF are measured in the aqueous humor sample.

60. The method of any one of aspects 57-59, wherein said treating the patient for the melanoma comprises performing surgery to excise the melanoma.

61. The method of aspect 60, wherein said treating further comprises performing radiation therapy or administering interferon, interleukin-2 (IL-2), dacarbazine, vemurafenib, dabrafenib, trametinib, pembrolizumab, ipilimumab, tremelimumab, nivolumab/relatlimab, or imiquimod, or a combination thereof.

62. The method of any one of aspects 57-61, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.

63. The method of aspect 62, wherein the aptamer-based proteomic assay is performed using a multiplex aptamer array.

64. The method of any one of aspects 57-63, wherein the subject has not yet developed clinical symptoms.

65. The method of any one of aspects 57-63, wherein the subject has developed clinical symptoms.

66. A method of monitoring melanoma in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein detection of increased levels of the one or more biomarkers selected from FSTL1, ENPP2, ANG, MET, and HGF in the second aqueous humor sample compared to the first aqueous humor sample indicate that the patient is worsening, and wherein detection of decreased levels of the one or more biomarkers selected from FSTL1, ENPP2, ANG, MET, and HGF in the second aqueous humor sample compared to the first aqueous humor sample indicate that the patient is improving.

67. A method of monitoring efficacy of a treatment of a patient for melanoma, the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF); and
evaluating the efficacy of the treatment, wherein detection of increased levels of the one or more biomarkers selected from FSTL1, ENPP2, ANG, MET, and HGF in the second aqueous humor sample compared to the first aqueous humor sample indicate that the patient is worsening or not responding to the treatment, and detection of decreased levels of the one or more biomarkers selected from FSTL1, ENPP2, ANG, MET, and HGF in the second aqueous humor sample compared to the first aqueous humor sample indicate that the patient is improving.

68. The method of aspect 67, further comprising altering the treatment if the patient is worsening or not responding to the treatment.

69. The method of any one of aspects 57-68, wherein the melanoma is choroidal melanoma.

70. A kit for diagnosing melanoma comprising agents for detecting at least 3 biomarkers selected from the group consisting of follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF).

71. The kit of aspect 70, wherein the kit comprises agents for detecting all of the FSTL1, ENPP2, ANG, MET, and HGF biomarkers.

72. The kit of aspect 70 or 71, further comprising reagents for performing an aptamer-based proteomic assay or immunoassay.

73. The kit of aspect 72, wherein the kit comprises an aptamer or antibody that specifically binds to FSTL1, an aptamer or antibody that specifically binds to ENPP2, an aptamer or antibody that specifically binds to ANG, an aptamer or antibody that specifically binds to MET, and an aptamer or antibody that specifically binds to HGF.

74. The kit of any one of aspects 70-73, further comprising instructions for determining whether a subject has melanoma.

75. A protein selected from the group consisting of follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) for use as a biomarker in diagnosing melanoma.

76. An in vitro method of diagnosing melanoma, the method comprising:
obtaining an aqueous humor sample from an eye of the patient; and
measuring levels of one or more biomarkers selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein increased levels of the one or more biomarkers selected from FSTL1, ENPP2, ANG, MET, and HGF compared to reference value ranges for the levels of the one or more biomarkers in a control aqueous humor sample indicate that the patient has melanoma.

77. The method of aspect 76, wherein the melanoma is choroidal melanoma.

78. A method of diagnosing and treating uveitis in a patient, the method comprising:
obtaining an aqueous humor sample from an eye of the patient;
measuring levels of one or more biomarkers selected from retinoic acid receptor responder 2 (RARRES2), biotinidase (BTD), cystatin C (CST3), TIMP metallopeptidase inhibitor 2 (TIMP2), and prostaglandin D2 synthase (PTGDS) in the aqueous humor sample, wherein increased levels of the one or more biomarkers selected from CST3 and TIMP2 compared to reference value ranges for the levels of the one or more biomarkers in a control aqueous humor sample indicate that the patient has uveitis; and
treating the patient for the uveitis if the patient has a positive diagnosis for uveitis.

78. A method of diagnosing and treating uveitis in a patient, the method comprising:
obtaining an aqueous humor sample from an eye of the patient;
measuring levels of one or more biomarkers selected from retinoic acid receptor responder 2 (RARRES2), biotinidase (BTD), cystatin C (CST3), TIMP metallopeptidase inhibitor 2 (TIMP2), and prostaglandin D2 synthase (PTGDS) in the aqueous humor sample, wherein increased levels of the one or more biomarkers selected from RARRES2, BTD, CST3, TIMP2, and PTGDS compared to reference value ranges for the levels of the one or more biomarkers in a control aqueous humor sample indicate that the patient has uveitis; and
treating the patient for the uveitis if the patient has a positive diagnosis for uveitis.

79. The method of aspect 78, wherein the levels of at least two, at least three, or at least four biomarkers selected from RARRES2, BTD, CST3, TIMP2, and PTGDS are measured in the aqueous humor sample.

80. The method of aspect 79, wherein the levels of RARRES2, BTD, CST3, TIMP2, and PTGDS are measured in the aqueous humor sample.

81. The method of any one of aspects 78-80, wherein said treating the patient for the uveitis comprises administering a glucocorticoid steroid, a cycloplegic agent, an antimetabolite, a T-cell inhibitor, an anti-tumor necrosis factor (TNF) agent, a biologic agent, an alkylating agent, an antibiotic for bacterial uveitis, an antiviral agent for viral uveitis, or an antifungal agent for fungal uveitis, or performing a vitrectomy, or a combination thereof.

82. The method of any one of aspects 78-81, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.

83. The method of aspect 82, wherein the aptamer-based proteomic assay is performed using a multiplex aptamer array.

84. The method of any one of aspects 78-83, wherein the subject has not yet developed clinical symptoms.

85. The method of any one of aspects 78-84, wherein the subject has developed clinical symptoms.

86. A method of monitoring uveitis in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected retinoic acid receptor responder 2 (RARRES2), biotinidase (BTD), cystatin C (CST3), TIMP metallopeptidase inhibitor 2 (TIMP2), and prostaglandin D2 synthase (PTGDS) in the aqueous humor sample, wherein detection of increased levels of the one or more biomarkers selected from RARRES2, BTD, CST3, TIMP2, and PTGDS in the second aqueous humor sample compared to the first aqueous humor sample indicate that the patient is worsening, and wherein detection of decreased levels of the one or more biomarkers selected from RARRES2, BTD, CST3, TIMP2, and PTGDS in the second aqueous humor sample compared to the first aqueous humor sample indicate that the patient is improving.

87. A method of monitoring efficacy of a treatment of a patient for uveitis, the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from retinoic acid receptor responder 2 (RARRES2), biotinidase (BTD), cystatin C (CST3), TIMP metallopeptidase inhibitor 2 (TIMP2), and prostaglandin D2 synthase (PTGDS); and
evaluating the efficacy of the treatment, wherein detection of increased levels of the one or more biomarkers selected from RARRES2, BTD, CST3, TIMP2, and PTGDS in the second aqueous humor sample compared to the first aqueous humor sample indicate that the patient is worsening or not responding to the treatment, and detection of decreased levels of the one or more biomarkers selected from RARRES2, BTD, CST3, TIMP2, and PTGDS in the second aqueous humor sample compared to the first aqueous humor sample indicate that the patient is improving.

88. The method of aspect 87, further comprising altering the treatment if the patient is worsening or not responding to the treatment.

89. A kit for diagnosing uveitis comprising agents for detecting at least 3 biomarkers selected from the group consisting of retinoic acid receptor responder 2 (RARRES2), biotinidase (BTD), cystatin C (CST3), TIMP metallopeptidase inhibitor 2 (TIMP2), and prostaglandin D2 synthase (PTGDS).

90. The kit of aspect 89, wherein the kit comprises agents for detecting all of the RARRES2, BTD, CST3, TIMP2, and PTGDS biomarkers.

91. The kit of aspect 89 or 90, further comprising reagents for performing an aptamer-based proteomic assay or immunoassay.

92. The kit of aspect 91, wherein the kit comprises an aptamer or antibody that specifically binds to RARRES2, an aptamer or antibody that specifically binds to BTD, an aptamer or antibody that specifically binds to CST3, an aptamer or antibody that specifically binds to TIMP2, and an aptamer or antibody that specifically binds to PTGDS.

93. The kit of any one of aspects 89-92, further comprising instructions for determining whether a subject has uveitis.

94. A protein selected from the group consisting of retinoic acid receptor responder 2 (RARRES2), biotinidase (BTD), cystatin C (CST3), TIMP metallopeptidase inhibitor 2 (TIMP2), and prostaglandin D2 synthase (PTGDS) for use as a biomarker in diagnosing uveitis.

95. An in vitro method of diagnosing uveitis, the method comprising:
obtaining an aqueous humor sample from an eye of the patient; and
measuring levels of one or more biomarkers selected from retinoic acid receptor responder 2 (RARRES2), biotinidase (BTD), cystatin C (CST3), TIMP metallopeptidase inhibitor 2 (TIMP2), and prostaglandin D2 synthase (PTGDS) in the aqueous humor sample, wherein increased levels of the one or more biomarkers selected from RARRES2, BTD, CST3, TIMP2, and PTGDS compared to reference value ranges for the levels of the one or more biomarkers in a control aqueous humor sample indicate that the patient has uveitis.

96. A method of diagnosing and treating neovascular inflammatory vitreoretinopathy (NIV) in a patient, the method comprising:
obtaining an aqueous humor sample from an eye of the patient;
measuring levels of one or more biomarkers selected from serpin family C member 1 (SERPINC1), hemopexin (HPX), coagulation factor II (F2), complement C9 (C9), and complement C6 (C6) in the aqueous humor sample, wherein increased levels of the one or more biomarkers selected from SERPINC1, HPX, F2, C9, and C6 compared to reference value ranges for the levels of the one or more biomarkers in a control aqueous humor sample indicate that the patient has neovascular inflammatory vitreoretinopathy (NIV); and
treating the patient for the neovascular inflammatory vitreoretinopathy (NIV) if the patient has a positive diagnosis for neovascular inflammatory vitreoretinopathy (NIV).

97. The method of aspect 96, wherein the levels of at least two, at least three, or at least four biomarkers selected from SERPINC1, HPX, F2, C9, and C6 are measured in the aqueous humor sample.

98. The method of aspect 97, wherein the levels of SERPINC1, HPX, F2, C9, and C6 are measured in the aqueous humor sample.

99. The method of any one of aspects 96-98, wherein said treating the patient for the neovascular inflammatory vitreoretinopathy comprises administering fluocinolone acetonide, dexamethasone, or bevacizumab, or performing panretinal scatter photocoagulation (PRP), vitrectomy, trabeculectomy, or a combination thereof.

100. The method of any one of aspects 96-99, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.

101. The method of aspect 100, wherein the aptamer-based proteomic assay is performed using a multiplex aptamer array.

102. The method of any one of aspects 96-101, wherein the subject has not yet developed clinical symptoms.

103. The method of any one of aspects 96-101, wherein the subject has developed clinical symptoms.

104. A method of monitoring neovascular inflammatory vitreoretinopathy (NIV) in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from serpin family C member 1 (SERPINC1), hemopexin (HPX), coagulation factor II (F2), complement C9 (C9), and complement C6 (C6) in the aqueous humor sample, wherein detection of increased levels of the one or more biomarkers selected from SERPINC1, HPX, F2, C9, and C6 in the second aqueous humor sample compared to the first aqueous humor sample indicate that the patient is worsening, and wherein detection of decreased levels of the one or more biomarkers selected from SERPINC1, HPX, F2, C9, and C6 in the second aqueous humor sample compared to the first aqueous humor sample indicate that the patient is improving.

105. A method of monitoring efficacy of a treatment of a patient for neovascular inflammatory vitreoretinopathy (NIV), the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from serpin family C member 1 (SERPINC1), hemopexin (HPX), coagulation factor II (F2), complement C9 (C9), and complement C6 (C6); and
evaluating the efficacy of the treatment, wherein detection of increased levels of the one or more biomarkers selected from SERPINC1, HPX, F2, C9, and C6 in the second aqueous humor sample compared to the first aqueous humor sample indicate that the patient is worsening or not responding to the treatment, and detection of decreased levels of the one or more biomarkers selected from SERPINC1, HPX, F2, C9, and C6 in the second aqueous humor sample compared to the first aqueous humor sample indicate that the patient is improving.

106. The method of aspect 105, further comprising altering the treatment if the patient is worsening or not responding to the treatment.

107. A kit for diagnosing neovascular inflammatory vitreoretinopathy (NIV) comprising agents for detecting at least 3 biomarkers selected from the group consisting of serpin family C member 1 (SERPINC1), hemopexin (HPX), coagulation factor II (F2), complement C9 (C9), and complement C6 (C6).

108. The kit of aspect 107, wherein the kit comprises agents for detecting all of the SERPINC1, HPX, F2, C9, and C6 biomarkers.

109. The kit of aspect 107 or 108, further comprising reagents for performing an aptamer-based proteomic assay or immunoassay.

110. The kit of aspect 109, wherein the kit comprises an aptamer or antibody that specifically binds to SERPINC1, an aptamer or antibody that specifically binds to HPX, an aptamer or antibody that specifically binds to F2, an aptamer or antibody that specifically binds to C9, and an aptamer or antibody that specifically binds to C6.

111. The kit of any one of aspects 107-110, further comprising instructions for determining whether a subject has NIV.

112. A protein selected from the group consisting of serpin family C member 1 (SERPINC1), hemopexin (HPX), coagulation factor II (F2), complement C9 (C9), and complement C6 (C6) for use as a biomarker in diagnosing neovascular inflammatory vitreoretinopathy (NIV).

113. An in vitro method of diagnosing neovascular inflammatory vitreoretinopathy (NIV), the method comprising:
obtaining an aqueous humor sample from an eye of the patient; and
measuring levels of one or more biomarkers selected from serpin family C member 1 (SERPINC1), hemopexin (HPX), coagulation factor II (F2), complement C9 (C9), and complement C6 (C6) in the aqueous humor sample, wherein increased levels of the one or more biomarkers selected from SERPINC1, HPX, F2, C9, and C6 compared to reference value ranges for the levels of the one or more biomarkers in a control aqueous humor sample indicate that the patient has neovascular inflammatory vitreoretinopathy (NIV).

114. A method of predicting biological age and determining risk of age-related pathology and morbidity in a patient, the method comprising:
obtaining an aqueous humor sample from an eye of the patient;
measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein decreased levels of the one or more biomarkers selected from MMP10, NRP1, SEMA3C, HES5, and FGFRL1 compared to reference value ranges for the levels of the one or more biomarkers in a control aqueous humor sample indicate that the patient has a risk of age-related pathology and morbidity; and
increasing screening of the patient for an aging-related disease if the patient is identified as having the risk age-related pathology and morbidity.

115. The method of aspect 114, further comprising administering a treatment for the aging-related disease to the patient if the patient is identified as having the aging-related disease.

116. A method of monitoring biological aging of an eye in a patient, the method comprising:
obtaining an aqueous humor sample from an eye of the patient; and
measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein levels of the one or more biomarkers selected from MMP10, NRP1, SEMA3C, HES5, and FGFRL1 are correlated with biological age of the eye.

117. The method of aspect 116, wherein the levels of at least two, at least three, or at least four biomarkers selected from MMP10, NRP1, SEMA3C, HES5, and FGFRL1 are measured in the aqueous humor sample.

118. The method of aspect 117, wherein the levels of MMP10, NRP1, SEMA3C, HES5, and FGFRL1 are measured in the aqueous humor sample.

119. The method of any one of aspects 116-118, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.

120. The method of aspect 119, wherein the aptamer-based proteomic assay is performed using a multiplex aptamer array.

121. A kit comprising agents for detecting at least 3 biomarkers selected from the group consisting of stromelysin-2 (MMP10), neuropilin-1 (NRP1), semaphorin 3C (SEMA3C), hes family bHLH transcription factor 5 (HES5), and fibroblast growth factor receptor like 1 (FGFRL1).

122. The kit of aspect 121, wherein the kit comprises agents for detecting all of the MMP10, NRP1, SEMA3C, HES5, and FGFRL1 biomarkers.

123. The kit of aspect 121 or 122, further comprising reagents for performing an aptamer-based proteomic assay or immunoassay.

124. The kit of aspect 123, wherein the kit comprises an aptamer or antibody that specifically binds to MMP10, an aptamer or antibody that specifically binds to NRP1, an aptamer or antibody that specifically binds to SEMA3C, an aptamer or antibody that specifically binds to HES5, and an aptamer or antibody that specifically binds to FGFRL1.

125. The kit of any one of aspects 121-124, further comprising instructions for predicting biological age and determining risk of age-related pathology and morbidity in a patient or determining the biological age of the eye.

126. A protein selected from the group consisting of stromelysin-2 (MMP10), neuropilin-1 (NRP1), semaphorin 3C (SEMA3C), hes family bHLH transcription factor 5 (HES5), and fibroblast growth factor receptor like 1 (FGFRL1) for use as a biomarker in predicting biological age and determining risk of age-related pathology and morbidity.

127. A protein selected from the group consisting of stromelysin-2 (MMP10), neuropilin-1 (NRP1), semaphorin 3C (SEMA3C), hes family bHLH transcription factor 5 (HES5), and fibroblast growth factor receptor like 1 (FGFRL1) for use as a biomarker for determining biological age of an eye.

128. An in vitro method of predicting biological age and determining risk of age-related pathology and morbidity, the method comprising:
obtaining an aqueous humor sample from an eye of the patient; and
measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein decreased levels of the one or more biomarkers selected from MMP10, NRP1, SEMA3C, HES5, and FGFRL1 compared to reference value ranges for the levels of the one or more biomarkers in a control aqueous humor sample indicate that the patient has a risk of age-related pathology and morbidity.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Methods for Monitoring Molecular Biomarkers for Aging and Disease

### Introduction

A common technique to increase proteomic depth is depleting the most abundant proteins to allow less abundant proteins to be accurately detected by the mass spectrometer.^{4,5} Here, we demonstrate that protein depletion of AH samples even impairs proteomic depth, since important proteins are removed along with high abundant proteins. In contrast, an aptamer-based proteomics assay with over 7,000 probes⁶ detected almost 2,000 novel proteins in healthy AH representing a manifold expansion of previously known AH proteins. These results reveal the molecular basis of known AH functions including immune response, hemostasis, and proteolysis and provide new insights into AH physiology, highlighting axon guidance-related processes as a previously unrecognized key molecular network in AH. In addition, this study uncovers age-related modulations of the AH proteome and implies a protein exchange between vitreous and AH, suggesting AH as a potential platform to better understand age-related morbidity and to monitor vitreoretinal diseases.

These results provide new avenues for future high-resolution studies on liquid biopsies with particularly low volume and low protein concentration and highlight AH as an easily accessible ocular specimen to monitor the entire ocular system.

### Results

### Protein Depletion Results in Reduced Protein Numbers and Impaired Detection of Less Abundant Proteins

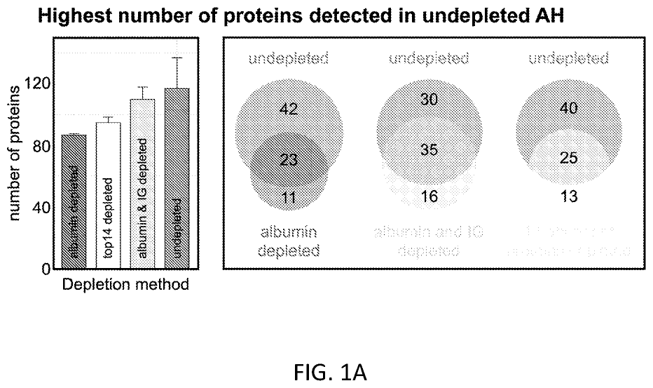

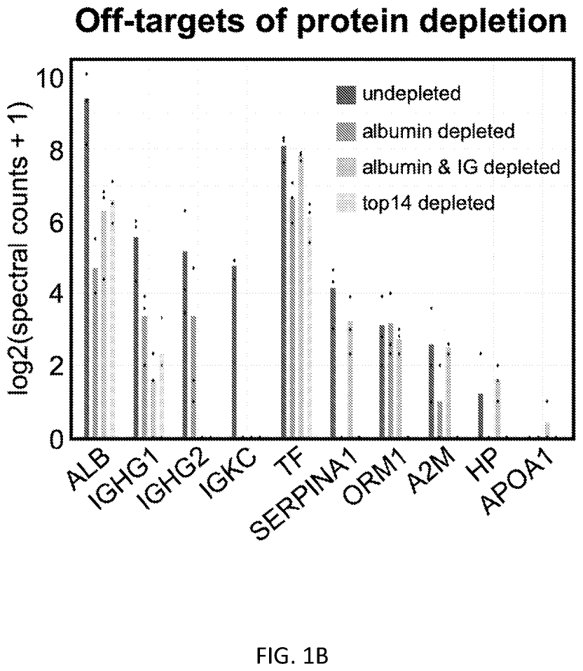

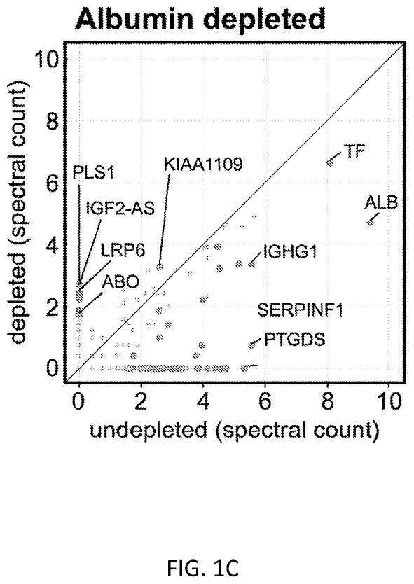

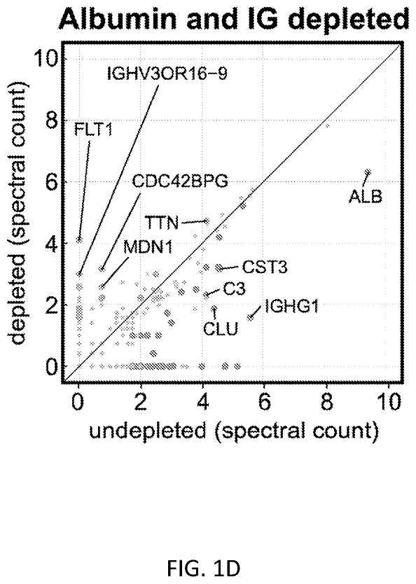

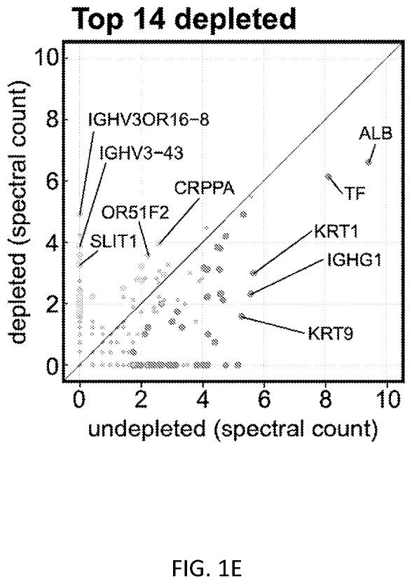

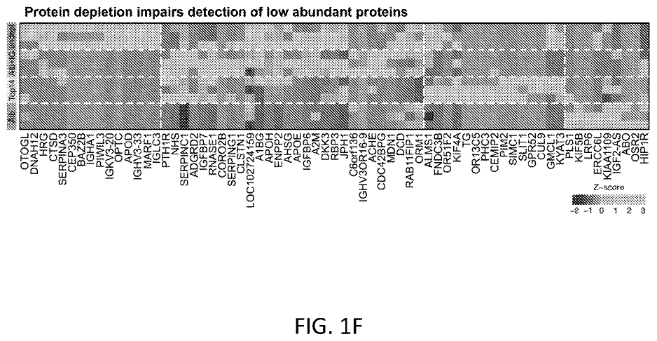

To investigate an effect of protein depletion on AH protein detection, AH samples from three patients were each divided in four parts and processed as undepleted samples as well as with three different depletion methods (i.e., depleting only albumins, albumins and immunoglobulins together, or the 14 most abundant plasma proteins) prior to analysis by LC-MS. The highest number of proteins was detected in undepleted samples (117.0±20.0; 65 unique proteins in all samples) compared to albumin depletion (87.0±1.0; 34 unique proteins in all samples), albumin and immunoglobulin depletion (110.0±8.0; 51 unique proteins in all samples), and 14 abundant protein depletion (95±3.6; 38 unique proteins in all samples) (FIG. 1A). Interestingly, the number of proteins which were exclusively detected in undepleted samples was higher than the number of proteins detected only after each of the three depletion methods (FIG. 1A). Each depletion method was able to substantially decrease its target protein levels, although significant off-target effects were also evident (FIG. 1B). For example, albumin depletion not only substantially decreased albumin levels, but also some of the most abundant proteins, among them immunoglobulins, such as immunoglobulin heavy constant gamma 1 (IGHG1), serotransferrin (TF) and alpha-1-antitrypsin (SERPINA1). Similarly, albumin and immunoglobulin depletion as well as 14 abundant protein depletion resulted in decreased levels of albumin and immunoglobulins, whereas alpha-1-acid glycoprotein 1 (ORM1) and alpha-2-macroglobulin (A2M) were only depleted by 14 abundant protein depletion. Analyzing differentially expressed proteins between albumin depleted and undepleted samples revealed that 51 proteins including pigment epithelium-derived factor (SERPINF1), which plays important roles in the diseased eye,⁷ were decreased or lost in albumin depleted samples, whereas only 8 proteins, among them plastin-1 (PLS1) were better detected after albumin depletion (FIG. 1C). Similarly, albumin and immunoglobulin depletion resulted in no or decreased detection of 35 proteins, such as complement C3 (C3), whereas 15 proteins, including vascular endothelial growth factor receptor 1 (FLT1), were identified more effectively in comparison to undepleted samples (FIG. 1D). Likewise, 21 proteins were preferentially identified after 14 abundant protein depletion, whereas 49 proteins were better detectable in undepleted samples (FIG. 1E). These findings demonstrate that different protein depletion methods differentially affect proteomic profiles of AH samples, including key proteins of the healthy and diseased eye being missed after protein depletion.

Additionally, we investigated whether protein depletion improves detection of low abundant proteins. Surprisingly, all 3 depletion methods tended to result in rather impaired detection of low abundant proteins with less than 10 mean spectral counts in each group (FIG. 1F). Of a total of 67 differentially expressed low abundant proteins, 43 (64.2%) were better or similarly detected in undepleted samples. There were only 15 proteins (22.4%) which were better detected after 14 abundant protein depletion, among them serine/threonine-protein kinase pim-2 (PIM2) and SUMO-interacting motif-containing protein 1 (SIMC1). Additionally, only 9 proteins (13.4%) were preferentially detected after albumin depletion, among them kinesin-1 heavy chain (KIF5B) and LRP6. 15 low abundant proteins were only detected in undepleted samples, among them cathepsin D (CTSD) and opticin (OPTC) (FIG. 1F). These results demonstrate that protein depletion of low volume liquid biopsies may impair detection of low abundant proteins, indicating that depletion might not be the optimal method to increase proteomic depth.

### Aptamer-Based Assay Detects Large Number of Previously Unknown Aqueous Humor Proteins and Suggests Age-Related Proteomic Signatures

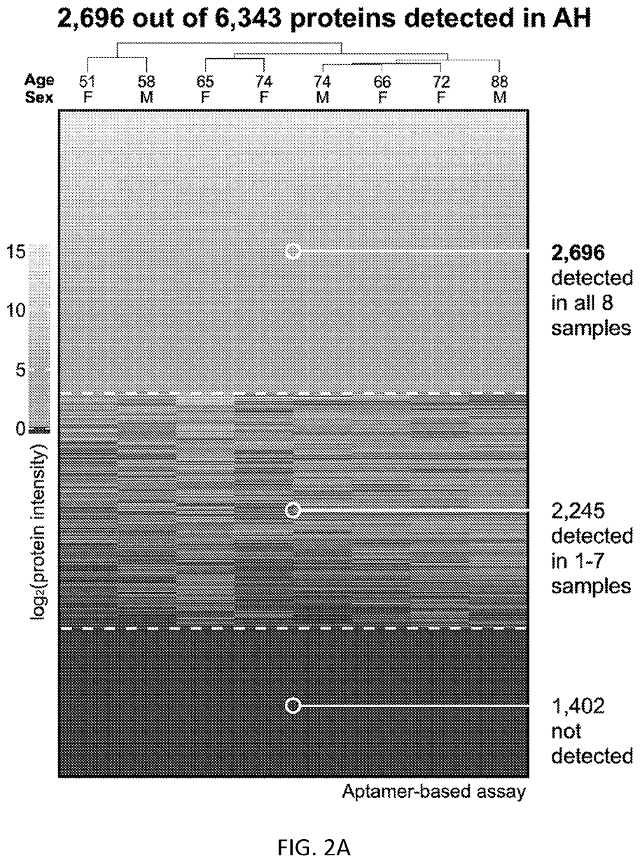

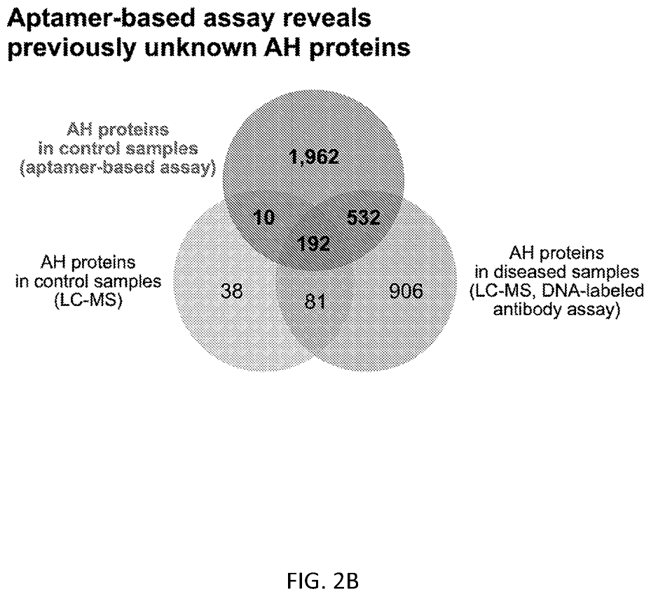

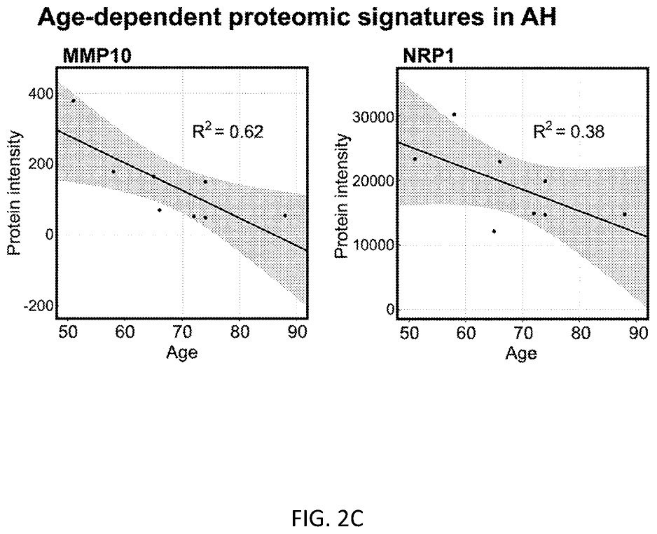

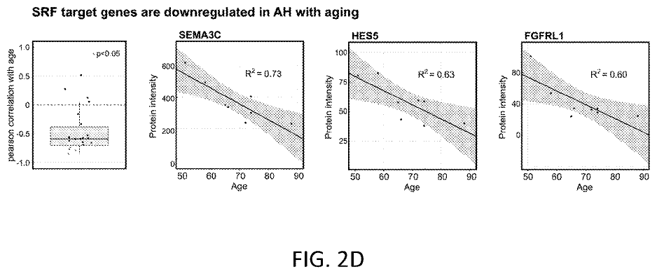

Proteomic depth in AH as determined by LC-MS was relatively low. Consequently, we next applied an aptamer-based assay, which is designed to detect 6,343 unique proteins and may be more appropriate to analyze very small sample volumes such as AH specimens.⁶ Indeed, the aptamer-based assay identified a total of 2,696 unique proteins in each of the 8 AH samples (FIG. 2A, Supplemental Table 1). In addition, 2,245 proteins were detected in at least 1 but not in all 8 samples.

We compared these 2,696 proteins with previously reported AH proteins from healthy as well as diseased samples, as determined by LC-MS (Supplemental Table 2). In relation to 321 reported AH proteins in control samples obtained from patients undergoing cataract surgery, the aptamer-based assay identified an 8.4-times higher number of proteins (n=2,696). 1,962 AH proteins (72.8% of all detected proteins) were only identified by the aptamer-based assay and were not reported in any of the available AH studies (FIG. 2B). This number even increased to 2,494 (92.5% of all detected proteins) when only studies on healthy AH samples were considered for comparison. 734 proteins (27.2% of all detected proteins) were identified by both the aptamer-based assay and LC-MS. In contrast, 1,025 AH proteins were reported in the literature but were not identified by the aptamer-based assay (FIG. 2C). The main reason for this discrepancy was that for 66.7% (n=684) of these proteins, no specific aptamer was included in the assay at the time of analysis. These unincluded proteins were mostly associated with mitochondrial energy production and immunoglobulin-associated processes (Supplemental FIG. 1). For the remaining 33.3% (n=341), a specific aptamer was available, but the signal was not above the limit of detection in each sample. However, it is worth noting that 227 of these 341 proteins were above the limit of detection in at least one sample, but not in all. In addition, the majority of these 341 proteins were discovered in studies investigating AH from diseased eyes (e.g. IL18, MMP12, and VEGFC⁸⁻¹⁰), which might explain why they were not detected in control AH samples. These results illustrate that the aptamer-based assay identified a large number of previously unknown AH proteins, and in addition, was able to detect a large proportion of known AH biomarkers for ocular diseases.

Age biomarkers have been shown to better predict age-related diseases and mortality than chronological age.¹¹ However, molecular age markers in the AH remain widely unexplored. In this study, heatmap-clustering of all detected AH proteins suggested an age-dependent trend pointing towards age-related proteomic signatures in the AH (FIG. 2A). A plethora of proteins with a significant association with patient age were identified, among them Neuropilin-1 (NRP1) and Stromelysin-2 (MMP10) (FIG. 2C and Supplemental FIG. 2A). Indeed, a strong decrease in MMP10 level was one of the previously observed age-related proteomic signatures in the blood.¹² Interestingly, recently it has been shown, that cerebrospinal fluid from young mice transferred to aged brains improved memory function.¹³ This process was mediated by serum response factor (SRF), and a downregulation of SRF target genes was observed in oligodendrocytes with age. We therefore investigated whether SRF target proteins are also age-regulated in the AH. Of 33 SRF target proteins included in the aptamer-based assay, 22 were identified in all AH samples (31 in 1-7 samples). Interestingly, 81.8% (n=18) of these proteins exhibited a negative Pearson correlation coefficient with patient age (FIG. 2D), among them semaphorin-3C (SEMA3C), transcription factor HES-5 (HES5), Fibroblast growth factor receptor-like 1 (FGFRL1), Calponin-2 (CNN2) and Protein delta homolog 2 (DLK2). We also investigated the cell type specific expression of the corresponding genes in the eye using published single cell RNA sequencing data. HES5 and FGFRL1 were predominantly expressed in retinal glia cells, whereas SEMA3C was most abundant in Schwann cells in the retinal pigment epithelium and iris, CNN2 in adaptive immune cells and DLK2 in retinal horizontal and amacrine cells (Supplement FIG. 2B). These results indicate that AH may serve to monitor the molecular age of the eye.

Aptamer-Based Assay Opens New Avenues for Insights into Aqueous Humor Physiology

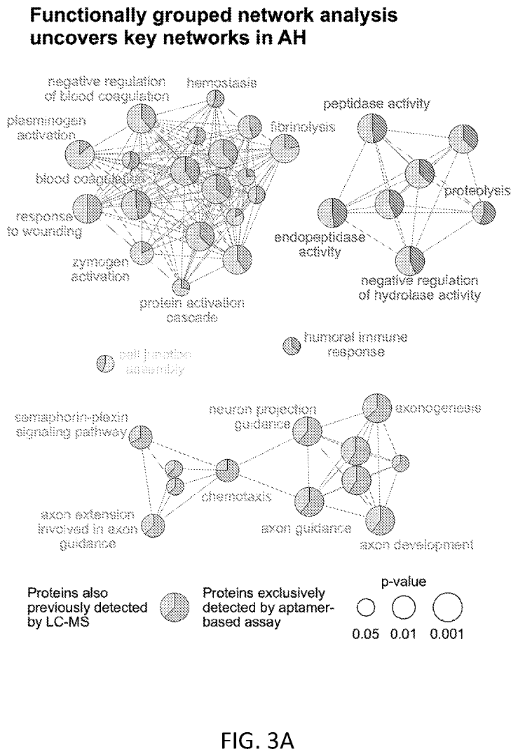

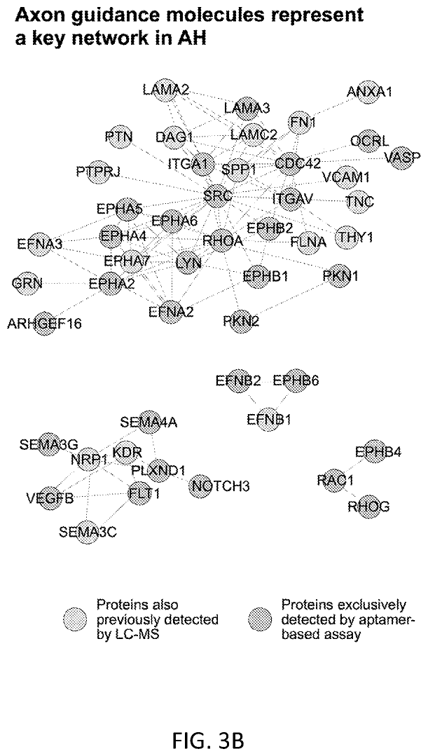

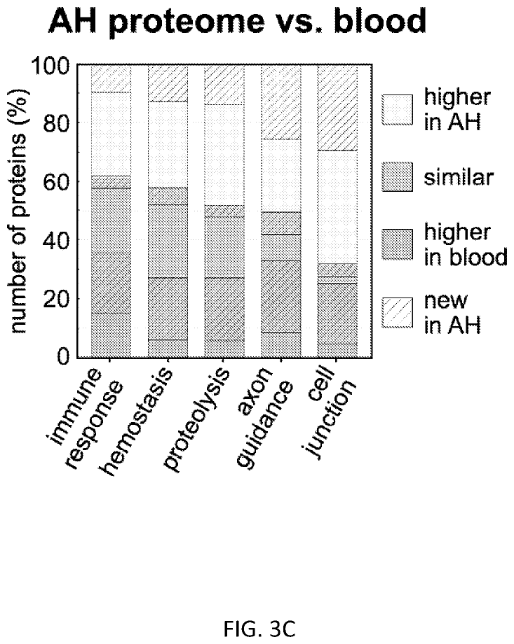

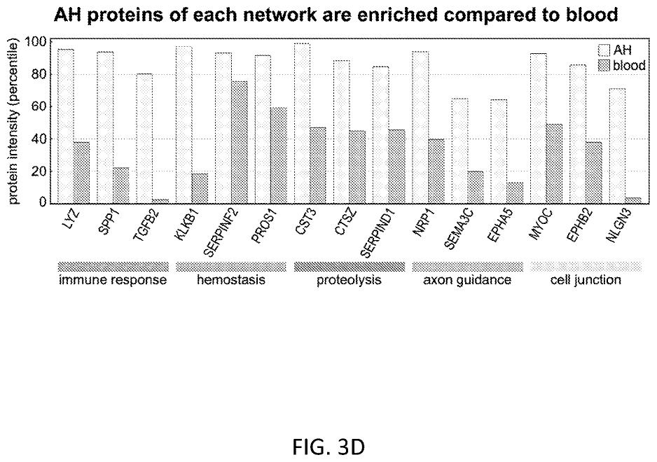

Immune surveillance and fibrinolytic processes are known AH functions, however little is known about which proteins are involved and whether other important AH pathways may exist. Based on increased proteomic resolution achieved by the aptamer-based assay, a functionally grouped network analysis provided new insights into AH physiology (FIG. 3A). Consistent with previous findings, the largest functionally grouped network comprised several hemostasis processes, including negative regulation of blood coagulation, fibrinolysis, and plasminogen activation.¹⁴ Other networks consisted of diverse proteolysis and humoral immune processes. Strikingly, one of the largest networks was composed of multiple axon guidance related processes, including chemotaxis and semaphorin-plexin signaling pathway, pointing towards a previously unrecognized key physiology in AH. Indeed, axon guidance proteins are involved in regulation of neuroinflammation and neovascular eye diseases further supporting a functional role in ocular pathology.^{15,16}

We next investigated which proportion of proteins in each network node had already been detected in AH in previous LC-MS studies and in contrast, how many proteins were exclusively identified using the aptamer-based assay. About 60% of the proteins associated to known AH pathways, including hemostasis (62.5%), proteolysis (55.5%), and immune (65.3%) networks have been previously detected in AH by LC-MS. However, several key proteins in these processes were newly identified using the aptamer-based assay, including vitamin K-dependent protein C (PROC), urokinase plasminogen activator surface receptor (PLAUR) and tissue factor pathway inhibitor (TFPI) in hemostasis, matrix metalloproteinase-9 (MMP9), kunitz-type protease inhibitor 1 (SPINT1) and tissue-type plasminogen activator (PLAT) in proteolysis, and membrane cofactor protein (CD46), complement factor H-related protein 4 (CFHR4) and triggering receptor expressed on myeloid cells 1 (TREM1) in humoral immune processes (Supplemental FIG. 3). Remarkably, the majority (62.8%, p<0.001 compared to all other groups of networks) of the axon guidance network proteins have previously not been recognized in AH omics studies using LC-MS but were now identified using the aptamer-based assay (FIG. 3A). We further analyzed the proteins involved in this network using a STRING interaction analysis (FIG. 3B). Several groups of axon guidance molecules were overrepresented in the top STRING subnetworks, among them ephrins (EFNA2, EFNA3, EFNB1, and EFNB2) and ephrin receptors (EPHA2, EPHA4, EPHA5, EPHA6, EPHA7, EPHB1, EPHB2, EPHB4, and EPHB6), as well as semaphorins (SEMA3C, SEMA3G, SEMA4A) and semaphorin receptors (PLXND1, NRP1). Most of these proteins, with a focus on the central network molecules, were identified for the first time in AH (FIG. 3B). At the same time, members of each of the aforementioned protein groups, among them EFNA3, EPHA7, SEMA3C and NRP1, have also been detected in AH by LC-MS before. These results further emphasize a key role of axon guidance molecules in AH and reveal that members of each protein group have been previously detected by LC-MS, however, the dominance of the network in AH has not been recognized, most likely due to limited proteomic resolution.

The AH specific environment was further demonstrated by comparing it to the blood proteome.¹⁷ 38.1%, 42.4%, and 48.4% of the proteins associated with the AH networks immune response, hemostasis, and proteolysis were more abundant in AH than in blood (FIG. 3C, Supplement Table 3). Strikingly, 50.7% of axon guidance proteins were detected at higher levels in AH than in blood, further emphasizing their central role in AH physiology. In addition, increased proteomic depth of the aptamer-based assay uncovered many previously unrecognized AH proteins which were even more abundant in blood, indicating that these proteins may infiltrate from plasma. Some of the specific AH proteins from each network were Lysozyme C (LYZ), Osteopontin (SPP1) and Transforming growth factor beta-2 proprotein (TGFB2) in immune response, Plasma kallikrein (KLKB1), Alpha-2-antiplasmin (SERPINF2) and protein S (PROS1) in hemostasis, Cystatin-C (CST3), Cathepsin Z (CTSZ) and Heparin cofactor 2 (SERPIND1) in proteolysis, NRP1, SEMA3C and EPHA5 in axon guidance, as well as Myocilin (MYOC), EPHB2 and Neuroligin-3 (NLGN3) in cell junction assembly (FIG. 3D). Interestingly, most of these AH-enriched proteins are associated with ocular physiology and pathology, including ocular immune privilege (TGFB2¹⁸), AMD and Alzheimer's disease (CST3¹⁹) and glaucoma (MYOC²⁰). These results further indicate a unique proteomic profile in AH which is clearly distinct from blood and reflects AH physiology.

### Aptamer-Based Assay Highlights Aqueous Humor as a Compelling Diagnostic Platform for Vitreoretinal Diseases

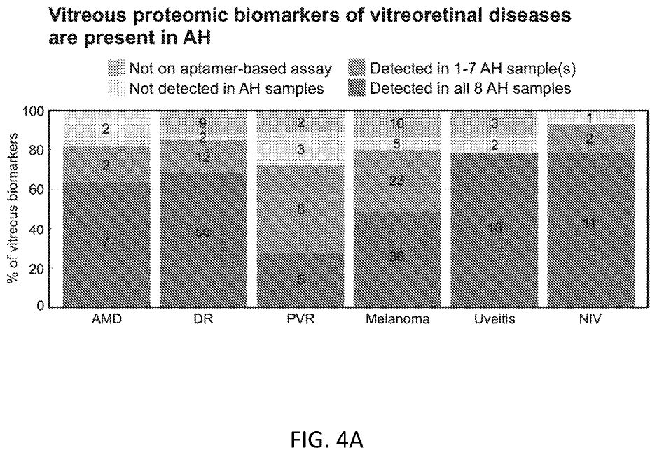

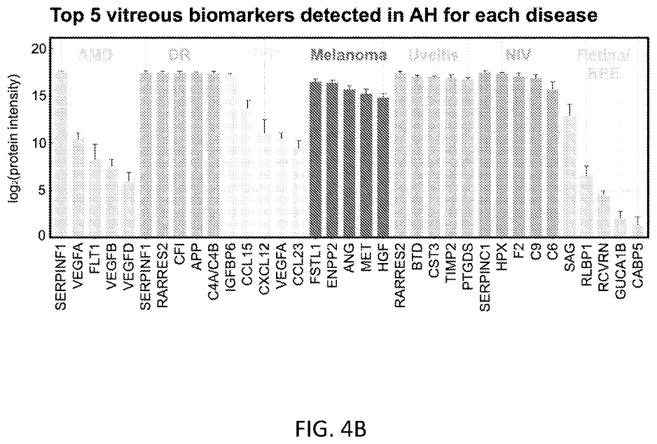

AH biopsy is significantly less invasive compared to vitreous biopsy and thus may represent an interesting alternative route of access for ocular liquid biopsies. Thus, we next investigated whether known vitreous biomarkers for diverse vitreoretinal diseases are also detectable in AH. This analysis revealed that the majority of vitreous biomarkers of age-related macular degeneration (AMD), diabetic retinopathy (DR), proliferative vitreoretinopathy (PVR), choroidal melanoma, uveitis, and neovascular inflammatory vitreoretinopathy (NIV) were indeed detected in AH (FIG. 4A, Supplementary Table 4). AMD biomarkers, such as SERPINF1, vascular endothelial growth factor A (VEGFA), and vascular endothelial growth factor receptor 1 (FLT1) were strongly detected in each of the analyzed AH samples (FIG. 4B), indicating that they are likely to be reliably detected in the AH of the diseased eye as well. Similarly, retinoic acid receptor responder protein 2 (RARRES2) and complement factor I (CFI) might serve as robust biomarkers for DR in the AH, insulin-like growth factor-binding protein 6 (IGFBP6) and C-C motif chemokine 15 (CCL15) for PVR, follistatin-related protein 1 (FSTL1) and hepatocyte growth factor (HGF) for choroidal melanoma, as well as cystatin-C (CST3) and metalloproteinase inhibitor 2 (TIMP2) for uveitis. Neuronal cell adhesion molecule (NrCAM) was previously found in the vitreous as a marker for autoimmune retinopathy and was found in all 8 AH samples at the 92^{nd} percentile of all detected proteins in this dataset.

In total, 156 out of 187 (83.4%) known vitreous biomarkers for vitreoretinal diseases were detected in at least one of the analyzed AH samples. In addition, numerous retina- and RPE-specific proteins such as s-arrestin (SAG), retinaldehyde-binding protein 1 (RLBP1) and recoverin (RCVRN) were also detected in significant quantities in the AH. These results indicate a substantial exchange of proteins between vitreous and AH and demonstrate that biomarkers of vitreoretinal diseases are generally detectable in the AH. Thus, AH liquid biopsy may represent a promising and considerably less invasive alternative acquisition route for liquid biopsies in the eye.

### Discussion

Liquid biopsies provide a revolutionary opportunity for personalized proteomics, allowing individualized molecular characterization and risk evaluation of the patient's disease, which may guide appropriate therapeutic strategy.²¹ However, in multiple fields such as in the human eye or in animal models, the collection and analysis of liquid biopsies is generally limited by factors such as small sample volumes and low protein concentrations. One potential solution might be protein depletion, in which the most abundant proteins are removed in order to improve the detection of less abundant proteins, and thus increase proteomic depth. This study compared 3 commonly used protein depletion kits and revealed that protein depletion on small volume samples obtained from the AH of the eye resulted in even lower protein numbers and impaired detection of low-abundant proteins, as determined by mass spectrometry (LC-MS). In contrast, an aptamer-based proteomics assay identified 2,696 unique proteins in the AH, corresponding to an 8.4-fold increase compared to previously reported LC-MS data. This study serves as the basis for upcoming high-resolution biomarker studies on small volume samples, including samples from AH. In addition, the results provide new insights into potential functions of AH, identify age-related AH proteins, and demonstrate that biomarkers of numerous ocular diseases, such as AMD or DR, can be detected in the AH.

Our experiments demonstrated that protein depletion of AH samples may be not an optimal method to increase proteomic depth. Apart from a decrease in target proteins, we also observed a decline in off-target proteins which are known to play important roles in the healthy and diseased eye, such as immunoglobulins (e.g. IGHG1) and pigment epithelium-derived factor (SERPINF1) after albumin depletion, or opticin (OPTC) and cathepsin D (CTSD) following all 3 depletion methods.^{7,22} Therefore, protein depletion may not be favorable to detect crucial proteins to study certain ocular diseases. Off-target protein depletion may be a consequence of protein binding to on-target proteins such as albumin,²³ an effect that may be further enhanced by low protein concentration in the AH, which is about 20 times lower than in plasma.²⁴ These findings provide a valuable foundation for future studies performing protein depletion on low volume samples such as AH.

As an alternative method to LC-MS-based proteomic detection in AH, we applied an aptamer-based proteomics assay, which identified 2,696 unique proteins in the AH, representing a 8.4-fold expansion of previously reported AH proteins.^{4,5,25-34} The aptamer-based assay is similar to a multiplex ELISA that is widely used in proteomics and which employs antibodies to detect a limited number of proteins. In contrast, the aptamer-based assay uses aptamers which are synthetic single-stranded nucleic acid-based molecules which specifically bind to their protein target and which are selected from large random sequence libraries.⁶ Aptamers having bound to their target protein are subsequently detected and quantified by PCR-based techniques, resulting in high sensitivity even for low abundant proteins.⁶ Thus, an aptamer-based assay might not only represent a beneficial alternative compared to LC-MS but also with regard to multiplex ELISA.

Functionally grouped network analysis was performed to address whether increased proteomic depth achieved by the aptamer-based assay could improve our understanding about AH physiology. Consistent with previous findings, the largest functionally grouped network comprised several hemostasis processes, while other networks consisted of diverse proteolysis and humoral immune processes.^{3,14} Several key molecules were identified in the AH immune response network, including proteins involved in ocular immune privilege (TGFB2, CD46¹⁸), AMD (CST3, SPP1, CFHR4^{19,35-37}), PVR (TREM1³⁸), and glaucoma (MYOC²⁰). PROS1 and KLKB1 were among the proteins associated to the AH hemostasis network and were previously reported to be involved in retinal neovascular pathology.^{39,40} Similarly, proteins of the AH proteolysis network, are known as risk genes for AMD and Alzheimer's disease (CST3¹⁹), and are involved in choroidal neovascularization (CTSZ⁴¹) and glaucoma (SERPIND1⁴²). These results further indicate a unique proteomic profile in AH which is clearly distinct from blood and reflects AH physiology. Remarkably, one of the largest functionally enriched networks was composed of numerous axon guidance related processes, involving multiple groups of axon guidance molecules, such as ephrins and semaphorins and their receptors. The majority of these proteins were previously missed by LC-MS in the AH but were now identified using the aptamer-based assay. These results suggest a key role of axon guidance molecules in AH and demonstrate that only high-resolution proteomic profiling using the aptamer-based assay paved the way to uncover this previously unrecognized aqueous humor protein network. Although members of each of the aforementioned protein groups have been previously detected in AH by LC-MS, the dominance of the network in AH has not been recognized before. This can likely be explained by the limited proteomic resolution of previous LC-MS studies compared to the aptamer-based assay. Although originally identified as molecules regulating axon growth in the developing nervous system, axon guidance molecules are involved in a number of processes, including tumorigenesis, angiogenesis, metabolic diseases, such as obesity and diabetic complications, as well as regulation of immune and inflammatory responses, particularly in neuroinflammation.^{15,43,44} Since the process of neuroinflammation plays a crucial role in various brain pathologies such as Alzheimer's and Parkinson's disease, but also in ocular conditions such as age-related macular degeneration,^{35,45,47} axon guidance molecules represent an interesting therapeutic target to enhance neuroprotection while attenuating neurodegeneration.¹⁵ In the eye, axon guidance molecules have been identified in the AH of patients with retinal vein occlusion or diabetic retinopathy,^{48,49} and have been shown to be involved in neovascular eye diseases.¹⁶ Mechanistically, the semaphorin/plexin pathway has been demonstrated to induce endothelial cell dysfunction, pericyte loss, vascular leakage as well as neovascularization in DR. Elevated levels of semaphorins in the AH of patients with DR were associated with impaired response to anti-VEGF therapy and in the animal model, inhibition of the pathway in combination with anti-VEGF therapy resulted in a synergistic anti angiogenic effect.⁴⁹ These findings further point towards a functional role of axon guidance molecules in ocular pathology. However, further studies are needed to verify the functional significance of overrepresentation of axon guidance molecules in AH.

Molecular age markers have been shown to reflect the physiological state of individuals and to better predict age-related diseases and mortality than chronological age.¹¹ Even though AH could be easily obtained from liquid biopsy or during cataract surgery, age biomarkers in the AH remain widely unexplored, currently limiting opportunities to monitor the molecular age of the eye. In this study, heatmap-clustering suggested an age-dependent trend among all detected AH proteins. Stromelysin-2 (MMP10) and neuropilin-1 (NRP1) were 2 of these proteins, exhibiting a decrease with age. Interestingly, MMP10 has been shown to play a beneficial role in immune response by alleviating the proinflammatory response of macrophages.⁵⁰ Since resident and infiltrating mononuclear cells are critically involved in the pathogenesis of many age-related ocular diseases,⁴⁵⁻⁴⁷ reduced MMP10 levels in AH of the elderly might contribute to an aggravated proinflammatory response contributing to augmented disease activity. Of note, a strong age-related decrease in MMP10 has also been observed in serum.¹² Another age-associated protein in the AH was NRP1, which also belongs to the group of axon guidance molecules. NRP1 regulates angiogenesis induced by various signals such as vascular endothelial growth factor and semaphorins. Therefore, NRP1 is considered a promising therapeutic target for the treatment of ocular diseases.¹⁶ However, no association between age and NRP1 expression in the eye has been reported so far, which might affect NRP1-targeting therapies. In addition, in glioblastoma cells, downregulation of NRP1 has been shown to be linked to resistance to anti-VEGF treatment.⁵¹ Anti-VEGF resistance is a common issue in patients with age-related macular degeneration and one risk factor is known to be advanced patient's age,⁵² which could possibly be related to the reduced NRP1 levels in elderly. Recently it has been shown, that cerebrospinal fluid transferred from young to old mice could rejuvenated the aged brain.¹³ Since this process was mediated by SRF and downregulation of SRF target genes was observed in oligodendrocytes with age, we next examined whether SRF target proteins were also age-regulated in the AH. Interestingly, 81.8% of the SRF target proteins identified in AH demonstrated a trend of decreasing abundance with age. Reanalysis of published single cell RNA sequencing data of the human eye revealed that these proteins, among them FGFRL1 and SEMA3C, were predominantly expressed in retinal glia, horizontal and amacrine cells, as well as in Schwann and adaptive immune cells. These results indicate that AH may serve to monitor the molecular age of the eye and suggest that SRF target molecules may represent interesting therapeutic targets for age-related pathologies not only of the brain but also of the eye.¹³ However, further studies are needed to validate these findings in larger cohorts and to address potential therapeutic applications.

Compared with vitreous biopsy, AH biopsy is much less invasive and could therefore represent an interesting alternative approach for liquid biopsies in the eye. Therefore, this study investigated whether vitreous biomarkers of various vitreoretinal diseases are also detectable in AH. Indeed, the majority of vitreous biomarkers of AMD, DR, PVR, choroidal melanoma, uveitis and NIV were identified by the aptamer-based assay in control AH. These results indicate that biomarkers of vitreoretinal diseases are generally detectable in the AH. However, this does not necessarily mean that these biomarkers are also differentially expressed in the AH of the diseased eye. Although 83.4% of known vitreous biomarkers for vitreoretinal diseases as well as retina- and RPE-specific proteins were detected in significant quantities in the AH, suggesting an exchange of proteins between AH and vitreous,⁵³ this question needs to be addressed in further studies involving AH from diseased eyes.

Compared to AH proteins previously detected by LC-MS, 341 proteins were not detected by the aptamer assay. However, protein detection is defined differently in the literature, with some studies accepting detection with a signal in only a part of the analyzed samples. In contrast, in this study, proteins were only considered detected if they were identified in each of the analyzed samples and it is worth noting that 227 of these 341 proteins were detected in at least one sample, but not in all. In addition, the majority of these 341 proteins were discovered in studies investigating AH from diseased eyes. Therefore, it may be possible that some of these proteins are only detectable in disease state and were therefore not detected in control AH samples analyzed in this study. The main reason why a protein was not detected was the absence of a specific aptamer, rather than insufficient sensitivity. These 684 proteins were most significantly enriched in mitochondrial energy related processes and immunoglobulin composition. Therefore, for future studies addressing these processes, the corresponding aptamers need to be added to the assay. Of note, several common retina-specific proteins such as Rhodopsin (RHO), Guanine nucleotide-binding protein G(T) subunit gamma-T1 (GNGT1) and Retinal rod rhodopsin-sensitive cGMP 3′,5′-cyclic phosphodiesterase subunit gamma (PDE6G) are currently not included in the aptamer assay, which may be relevant for eye-related research questions. However, since additional aptamers can be added to the assay, missing proteins may be included for future studies based on the results of this work.

Even though our study revealed 1,962 unique proteins that were previously not observed in AH, we acknowledge that this study is limited by a relatively small sample size. Particularly, regarding the identified age-associated AH proteins, further studies with larger sample sizes are required to validate our findings. In addition, the AH samples investigated in this study were obtained from cataract patients, thus an influence of cataract on the AH proteome cannot be completely excluded. However, due to ethical reasons, obtaining AH from completely healthy eyes is not feasible, making cataracts the most physiological condition accessible in clinical practice. In the literature, control AH is also obtained from cataract patients, which improves comparability with the data obtained in this study.^{4,5,25,26}

In conclusion, this study demonstrates that an aptamer-based proteomic assay can detect 2,696 unique proteins in aqueous humor, corresponding to a significant expansion compared to previously reported AH proteins. These results pave the way for future high-resolution biomarker studies on fluid samples with particularly low volume and low protein concentration, such as samples from aqueous humor or animal models. In addition, the results provide new insights into aqueous humor physiology, identify age-related aqueous humor proteins, and indicate a protein exchange between vitreous and aqueous humor that may allow to monitor various biomarkers of vitreoretinal diseases in the AH.

### Methods

Study approval. The study protocol was approved by the Institutional Review Board for Human Subjects Research (IRB) at Stanford University, was HIPAA compliant, and adhered to the tenets of the Declaration of Helsinki. All subjects underwent informed consent for study participation.

### Aqueous Sample Collection

Aqueous humor biopsies were collected from patients undergoing cataract surgery. After the eye was prepped and draped for surgery, the first step was to draw up at least 0.1 cc of aqueous through the clear cornea via a 30-gauge needle on a TB syringe. Immediately, the sample was sterilely transferred into a vial which was placed on dry ice. Then the samples were labelled and taken in a batch to the −80C freezer. These samples were separated to be analyzed by two different methods: LC-MS (n=3) and aptamer-based assay (n=9). Anterior chamber paracentesis was performed using a 31-gauge needle and approximately 100 microliters of undiluted aqueous humor were manually aspirated into a 1 cubic centimeter syringe. Samples were immediately stored at −80° C. until further analysis with LC-MS or an aptamer-based assay.

### Sample Depletion

AH samples analyzed by LC-MS were depleted of highly abundant proteins. 10 μl aliquots of each sample were used in each of the three depletion kits: Pierce™ Albumin Depletion Kit (Thermo Fisher Scientific, catalog number: 85160) for albumin only depletion, High Select™ HSA/Immunoglobulin Depletion Mini Spin Columns (Thermo Fisher Scientific, catalog number: A36365) for albumin/immunoglobulin depletion, and High Select™ Top14 Abundant Protein Depletion Mini Spin Columns (Thermo Fisher Scientific, catalog number: A36369) for 14 abundant protein depletion. Samples were processed following the manufacturer's instructions.

### Protein Quantification

Protein quantification of each sample was conducted with the Pierce™ BCA Protein Assay Kit (Thermo Fisher Scientific, catalog number: 23225) following the company provided protocol. The absorbance at 562 nm was measured using a Tecan Spark® Multimode Microplate Reader, and protein concentrations were calculated based on each absorbance value. Using the results of the BCA, aliquots containing at least 5 μg of total protein (sample volumes between 20 μl and 112 μl; mean: 52.3, sd: 29.7) were frozen at −80° C. until analysis by LC-MS.

### Liquid Chromatography-Tandem Mass Spectrometry

Aqueous humor samples were brought to a total volume of 100 μl with 50 mM triethylammonium bicarbonate. Samples were reduced in 10 mM Dithiothreitol for 30 minutes at 55° C. Following reduction, proteins were alkylated in 30 mM acrylamide at room temperature for 30 minutes. Digestion was performed using 0.5 μg of Trypsin/LysC protease (Promega) with overnight incubation at 37° C. The digestion was then quenched by addition of 1 μL of 50% formic acid in water, and immediately followed by desalting with C18 spin columns (GL biosciences). The desalted peptides were then dried down on a speed-vac, and the dried peptides were reconstituted in a final reconstitution buffer (2% acetonitrile with 0.1% formic acid in water) for LC-MS acquisition.

Mass spectrometry-based experiments were performed on a Q Exactive HF-X Hybrid Quadrupole-Orbitrap mass spectrometer (Thermo Scientific, San Jose, CA) attached to a nanoACQUITY UPLC system (Waters Corporation, Milford, MA) RRID:SCR_018703. The UPLC system was set to a flow rate of 300 nL/min, where mobile phase A was 0.2% formic acid in water and mobile phase B was 0.2% formic acid in acetonitrile. The analytical column was prepared in-house with an I.D. of 100 microns pulled to a nanospray emitter using a P2000 laser puller (Sutter Instrument, Novato, CA). The column was packed with NanoLCMS solutions 1.8 micron C18 stationary phase to a length of approximately 25 cm. Peptides were directly injected into the column with a gradient of 2-45% mobile phase B, followed by a high-B wash over a total of 80 minutes. The mass spectrometer was operated in a data dependent fashion using HCD fragmentation for MS/MS spectra generation.

The RAW data were analyzed using Byonic v4.1.5 (Protein Metrics, Cupertino, CA) to identify peptides and infer proteins. A concatenated FASTA file containing the Uniprot Homo sapiens sequences and other likely contaminants and impurities was used. Proteolysis with Trypsin/LysC was assumed to be semi-specific allowing for N-ragged cleavage with up to two missed cleavage sites. Both precursor and fragment mass accuracies were held within 12 ppm. Cysteine modified with propionamide was set as a fixed modification in the search. Variable modifications included oxidation on methionine, histidine and tryptophan, dioxidation on methionine and tryptophan, deamidation on asparagine and glutamine, methylation on lysine and arginine, and acetylation on protein N-terminus. Proteins were held to a false discovery rate of 1% using standard reverse-decoy technique.⁵⁴

### Aptamer-Based Assay Human Samples

A volume of 55 μl of each AH sample was analyzed with an aptamer-based proteomic assay (SomaScan® Assay, SomaLogic) with previously described specifications.^{6,55} Briefly, the assay utilizes a 96-well plate that contains a solution of ~7,000 slow off-rate modified aptamers (SOMAmer reagents), each of which are tagged with a 5′ fluorophore, a photocleavable linker, and biotin. Each reagent is bound to streptavidin-coated beads via biotin-streptavidin interactions. Aqueous samples (n=9) were added to each well to forming SOMAmer-protein complexes. Exposure to ultraviolet light cleaves the photocleavable linker and releases the complex from the beads back into the solution. The non-specific complexes, with much greater off rates, dissociate while the specific complexes remain bound. Then, the complexes are incubated in a buffer containing a polyanionic competitor to selectively disrupt and inhibit the reformation of nonspecific SOMAmer-protein complexes. A second set of streptavidin-coated beads captures the SOMAmer reagent-bound biotinylated proteins. The proteins are then denatured, and the SOMAmer reagents are released, leaving the biotinylated proteins bound to the beads. The released SOMAmer reagents are hybridized to complementary sequences on a microarray chip to measure tagged. The fluorescence intensity for each reagent correlates to the amount of available epitope (the SOMAmer reagent binding site) in the original sample and represents protein intensity.

### Bioinformatics

Mass spectrometry data were initially processed in RStudio (version 2022.02.0+443, R version 4.1.2). Gene symbols and organism were determined based on the UniProt database⁵⁶ and proteins were filtered for human proteins. The spectral counts for different isoforms of the same protein were merged for each gene symbol. After principal component analysis, differentially expressed proteins were determined using the limma package⁵⁷ with default parameters except using method=“robust” in ImFit. P-values were corrected for multiple testing using Benjamini-Hochberg adjustment implemented in the limma package. Proteins with log 2 fold change (log 2FC) >2 or <−2 and adjusted p value <0.05 were considered as differentially expressed proteins (DEP). Heatmaps were created with the R package ComplexHeatmap (version 2.10.0).⁵⁸ Gene ontology analysis and its visualization with dotplots and cnetplots were performed using the R package clusterProfiler (version 4.2.2).⁵⁹ Other data visualization was done using the ggplot2 package (version 3.3.5).

Aptamer-based assay data were normalized by Somalogic, as described previously.⁶⁰ Normalized data were imported to R Studio as described above. One of the 9 samples was excluded due to a strongly abnormal Hybridization Scale Factor. Aptamers' target annotation and mapping to UniProt accession numbers as well as Entrez gene identifiers were provided by Somalogic. Only human protein targets were retained for subsequent analysis (7,289 out of the 7,596 aptamers). The estimated limit of detection (eLoD) was calculated for each aptamer using a ‘robust estimate’ method as previously described.⁶⁰ Briefly, it was calculated as the median plus 4.9×median absolute deviation signal of the three buffer samples. The eLoD was then subtracted from each intensity value of each aptamer in each sample to obtain the actual protein intensity above the detection limit and values below 0 were replaced by 0. For some proteins, Somalogic provides more than one aptamer. In these cases (611 of expressed proteins) only the aptamer with the highest intensity was retained. Proteins were considered expressed if they were detected in each sample unless otherwise indicated. Functional enrichment analysis was performed with g:Profiler as previously specified.^{61,62} Briefly, a list with all identified aqueous humor proteins ranked according to their intensity values was uploaded to the g:Profiler website. The analysis was run with the following specifications: ordered query, filters on gene annotation data to Gene ontology biological processes and Reactome molecular pathways, no electronic GO annotations, size of functional category to min=15 and max=350. In addition, the background was customized to all proteins included in the aptamer-based assay.⁶² Functional groups of enrichment results were determined and visualized using ClueGO⁶³ in Cytoscape.⁶⁴ Protein-protein interactions were analyzed using the Cytoscape StringApp⁶⁵ with the following specifications: interaction score >0.9 and network type: full STRING network. The network was clustered using MCL cluster (granularity parameter: 2) and the top clusters were retained.

Aptamer-based assay data were also compared to previously published proteome datasets of aqueous humor, vitreous body or blood. Lists of proteins were extracted from each publication. When gene symbols were provided, they were filtered for approved symbols using the HGNC (Human Genome Organization Gene Nomenclature Committee) database.⁶⁶ If no gene symbols were provided in the original publication, symbols were obtained from UniProt database⁵⁶ and filtered for approved symbols using the HGNC database.⁶⁶ Disease-associated proteins identified in the vitreous of patients with age-related macular degeneration (AMD), diabetic retinopathy (DR), proliferative vitreoretinopathy (PVR), choroidal melanoma, uveitis and neovascular inflammatory vitreoretinopathy (NIV) were extracted from the original publications.^{1,7,38,67-69} In addition, proteins identified using LC-MS in the aqueous humor of patients with cataract,^{4,5,25,26} AMD,²⁷ DR,²⁸ Coat's disease,²⁹ and glaucoma,³⁰⁻³³ as well as proteins identified using DNA-labeled antibodies (Olink) in the aqueous humor of patients with uveal melanoma were obtained.³⁴ To compare the AH proteome with blood, the blood proteomic profile of healthy patients was reanalyzed.¹⁷

To investigate the cell type specific expression of the SRF target genes in the eye, we reanalyzed published single cell RNA sequencing data of human retina⁷⁰ (data from supplementary table 3), RPE/choroid⁷¹ (data from SI Data 1), cornea⁷² and ciliary body 73 (download from singlecell.broadinstitute.org). For ciliary body, the following clusters were combined to one cell type: ciliary body cells, COL9A1-hi ciliary body cells, CRYAA-hi ciliary body cells, pigmented ciliary body cells to ciliary body cells and fibroblasts, MEG3-hi fibroblasts, MGP-hi fibroblasts, ribosomal genes-hi fibroblasts, WIF1-hi fibroblasts to fibroblasts as well as cytotoxic T cells activated T cells to T cells. Mean expression for each cell type was calculated for each gene. Expression data were combined and normalized using DESeq2⁷⁴ in R.

### REFERENCES

1 Velez, G. et al. Liquid biopsy proteomics of uveal melanoma reveals biomarkers associated with metastatic risk. Mol Cancer 20, 39, doi:10.1186/s12943-021-01336-4 (2021).
2 Ignatiadis, M., Sledge, G. W. & Jeffrey, S. S. Liquid biopsy enters the clinic-implementation issues and future challenges. Nat Rev Clin Oncol 18, 297-312, doi:10.1038/s41571-020-00457-x (2021).
3 Goel, M., Picciani, R. G., Lee, R. K. & Bhattacharya, S. K. Aqueous humor dynamics: a review. Open Ophthalmol J 4, 52-59, doi:10.2174/1874364101004010052 (2010).
4 Bennett, K. L. et al. Proteomic analysis of human cataract aqueous humour: Comparison of one-dimensional gel LCMS with two-dimensional LCMS of unlabelled and iTRAQ®-labelled specimens. J Proteomics 74, 151-166, doi:10.1016/j.jprot.2010.10.002 (2011).
5 Richardson, M. R. et al. Proteomic analysis of human aqueous humor using multidimensional protein identification technology. Mol Vis 15, 2740-2750 (2009).
6 Gold, L. et al. Aptamer-based multiplexed proteomic technology for biomarker discovery. PLoS One 5, e15004, doi:10.1371/journal.pone.0015004 (2010).
7 Kersten, E. et al. Systemic and ocular fluid compounds as potential biomarkers in age-related macular degeneration. Surv Ophthalmol 63, 9-39, doi:10.1016/j.survophthal.2017.05.003 (2018).
8 Cabral, T. et al. Bevacizumab Injection in Patients with Neovascular Age-Related Macular Degeneration Increases Angiogenic Biomarkers. Ophthalmol Retina 2, 31-37, doi:10.1016/j.oret.2017.04.004 (2018).
9 Zhao, B. et al. Expression profile of IL-1 family cytokines in aqueous humor and sera of patients with HLA-B27 associated anterior uveitis and idiopathic anterior uveitis. Exp Eye Res 138, 80-86, doi:10.1016/j.exer.2015.06.018 (2015).
10 Markiewicz, L. et al. Altered Expression Levels of MMP1, MMP9, MMP12, TIMP1, and IL-1 beta as a Risk Factor for the Elevated IOP and Optic Nerve Head Damage in the Primary Open-Angle Glaucoma Patients. Biomed Res Int 2015, 812503, doi:10.1155/2015/812503 (2015).
11 Rivero-Segura, N. A., Bello-Chavolla, O. Y., Barrera-Vazquez, O. S., Gutierrez-Robledo, L. M. & Gomez-Verjan, J. C. Promising biomarkers of human aging: In search of a multi-omics panel to understand the aging process from a multidimensional perspective. Ageing Res Rev 64, 101164, doi:10.1016/j.arr.2020.101164 (2020).
12 Komosinska-Vassev, K. et al. Age- and gender-dependent changes in connective tissue remodeling: physiological differences in circulating MMP-3, MMP-10, TIMP-1 and TIMP-2 level. Gerontology 57, 44-52, doi:10.1159/000295775 (2011).
13 Iram, T. et al. Young CSF restores oligodendrogenesis and memory in aged mice via Fgf17. Nature 605, 509-515, doi:10.1038/s41586-022-04722-0 (2022).
14 Sugioka, K., Fukuda, K., Nishida, T. & Kusaka, S. The fibrinolytic system in the cornea: A key regulator of corneal wound healing and biological defense. Exp Eye Res 204, 108459, doi:10.1016/j.exer.2021.108459 (2021).

1. A method of diagnosing and treating age-related macular degeneration (AMD) in a patient, the method comprising:
obtaining an aqueous humor sample from an eye of the patient;
measuring levels of one or more biomarkers selected from serpin family F member 1 (SERPINF1), vascular endothelial growth factor A (VEGFA), fins related receptor tyrosine kinase 1 (FLT1), vascular endothelial growth factor B (VEGFB), and vascular endothelial growth factor D (VEGFD) in the aqueous humor sample, wherein increased levels of the one or more biomarkers selected from SERPINF1, VEGFA, FLT1, VEGFB, and VEGFD compared to reference value ranges for the levels of the one or more biomarkers in a control aqueous humor sample indicate that the patient has AMD; and
treating the patient for the AMD if the patient has a positive diagnosis for AMD.

2. (canceled)

3. The method of claim 1, wherein the levels of SERPINF1, VEGFA, FLT1, VEGFB, and VEGFD are measured in the aqueous humor sample.

4. The method of claim 1, wherein said treating the patient for the AMD comprises administering vitamin C, zinc, vitamin E, copper, beta-carotene, lutein, zeaxanthin, ranibizumab, aflibercept, brolucizumab, or faricimab, or a combination thereof to the patient.

5-18. (canceled)

19. A method of diagnosing and treating diabetic retinopathy in a patient, the method comprising:
obtaining an aqueous humor sample from an eye of the patient;
measuring levels of one or more biomarkers selected from serpin family F member 1 (SERPINF1), retinoic acid receptor responder 2 (RARRES2), complement factor I (CFI), amyloid beta precursor protein (APP), complement C4A (C4A), and complement C4B (C4B) in the aqueous humor sample, wherein increased levels of the one or more biomarkers selected from SERPINF1, RARRES2, CFI, APP, C4A, and C4B compared to reference value ranges for the levels of the one or more biomarkers in a control aqueous humor sample indicate that the patient has diabetic retinopathy; and
treating the patient for the diabetic retinopathy if the patient has a positive diagnosis for diabetic retinopathy.

20. (canceled)

21. The method of claim 19, wherein the levels of SERPINF1, RARRES2, CFI, APP, C4A, and C4B are measured in the aqueous humor sample.

22. The method of claim 19, wherein said treating the patient for the diabetic retinopathy comprises administering an anti-vascular endothelial growth factor (VEGF) agent or a steroid, or performing panretinal laser photocoagulation or a vitrectomy, or a combination thereof.

23-38. (canceled)

39. A method of diagnosing and treating proliferative vitreoretinopathy (PVR) in a patient, the method comprising:
obtaining an aqueous humor sample from an eye of the patient;
measuring levels of one or more biomarkers selected from insulin like growth factor binding protein 6 (IGFBP6), C-C motif chemokine ligand 15 (CCL15), C-X-C motif chemokine ligand 12 (CXCL12), vascular endothelial growth factor A (VEGFA), and C-C motif chemokine ligand 23 (CCL23) in the aqueous humor sample, wherein increased levels of the one or more biomarkers selected from IGFBP6, CCL15, CXCL12, VEGFA, and CCL23 compared to reference value ranges for the levels of the one or more biomarkers in a control aqueous humor sample indicate that the patient has PVR; and
treating the patient for the PVR if the patient has a positive diagnosis for PVR.

40. (canceled)

41. The method of claim 39, wherein the levels of IGFBP6, CCL15, CXCL12, VEGFA, and CCL23 are measured in the aqueous humor sample.

42. The method of claim 39, wherein said treating the patient for the PVR comprises performing vitreous surgery, vitrectomy, membrane peeling, or retinotomy.

43-56. (canceled)

57. A method of diagnosing and treating melanoma in a patient, the method comprising:
obtaining an aqueous humor sample from an eye of the patient;
measuring levels of one or more biomarkers selected from follistatin like 1 (FSTL1), ectonucleotide pyrophosphatase/phosphodiesterase 2 (ENPP2), angiogenin (ANG), MET proto-oncogene, receptor tyrosine kinase (MET), and hepatocyte growth factor (HGF) in the aqueous humor sample, wherein increased levels of the one or more biomarkers selected from FSTL1, ENPP2, ANG, MET, and HGF compared to reference value ranges for the levels of the one or more biomarkers in a control aqueous humor sample indicate that the patient has melanoma; and
treating the patient for the melanoma if the patient has a positive diagnosis for melanoma.

58. (canceled)

59. The method of claim 57, wherein the levels of FSTL1, ENPP2, ANG, MET, and HGF are measured in the aqueous humor sample.

60. The method of claim 57, wherein said treating the patient for the melanoma comprises performing surgery to excise the melanoma.

61. The method of claim 60, wherein said treating further comprises performing radiation therapy or administering interferon, interleukin-2 (IL-2), dacarbazine, vemurafenib, dabrafenib, trametinib, pembrolizumab, ipilimumab, tremelimumab, nivolumab/relatlimab, or imiquimod, or a combination thereof.

62-77. (canceled)

78. A method of diagnosing and treating uveitis in a patient, the method comprising:
obtaining an aqueous humor sample from an eye of the patient;
measuring levels of one or more biomarkers selected from retinoic acid receptor responder 2 (RARRES2), biotinidase (BTD), cystatin C (CST3), TIMP metallopeptidase inhibitor 2 (TIMP2), and prostaglandin D2 synthase (PTGDS) in the aqueous humor sample, wherein increased levels of the one or more biomarkers selected from RARRES2, BTD, CST3, TIMP2, and PTGDS compared to reference value ranges for the levels of the one or more biomarkers in a control aqueous humor sample indicate that the patient has uveitis; and
treating the patient for the uveitis if the patient has a positive diagnosis for uveitis.

79. (canceled)

80. The method of claim 78, wherein the levels of RARRES2, BTD, CST3, TIMP2, and PTGDS are measured in the aqueous humor sample.

81. The method of claim 78, wherein said treating the patient for the uveitis comprises administering a glucocorticoid steroid, a cycloplegic agent, an antimetabolite, a T-cell inhibitor, an anti-tumor necrosis factor (TNF) agent, a biologic agent, an alkylating agent, an antibiotic for bacterial uveitis, an antiviral agent for viral uveitis, or an antifungal agent for fungal uveitis, or performing a vitrectomy, or a combination thereof.

82-95. (canceled)

96. A method of diagnosing and treating neovascular inflammatory vitreoretinopathy (NIV) in a patient, the method comprising:
obtaining an aqueous humor sample from an eye of the patient;
measuring levels of one or more biomarkers selected from serpin family C member 1 (SERPINC1), hemopexin (HPX), coagulation factor II (F2), complement C9 (C9), and complement C6 (C6) in the aqueous humor sample, wherein increased levels of the one or more biomarkers selected from SERPINC1, HPX, F2, C9, and C6 compared to reference value ranges for the levels of the one or more biomarkers in a control aqueous humor sample indicate that the patient has neovascular inflammatory vitreoretinopathy (NIV); and
treating the patient for the neovascular inflammatory vitreoretinopathy (NIV) if the patient has a positive diagnosis for neovascular inflammatory vitreoretinopathy (NIV).

97. (canceled)

98. The method of claim 96, wherein the levels of SERPINC1, HPX, F2, C9, and C6 are measured in the aqueous humor sample.

99. The method of claim 96, wherein said treating the patient for the neovascular inflammatory vitreoretinopathy comprises administering fluocinolone acetonide, dexamethasone, or bevacizumab, or performing panretinal scatter photocoagulation (PRP), vitrectomy, trabeculectomy, or a combination thereof.

100-113. (canceled)

114. A method of predicting biological age and determining risk of age-related pathology and morbidity in a patient, the method comprising:
obtaining an aqueous humor sample from an eye of the patient;
measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein decreased levels of the one or more biomarkers selected from MMP10, NRP1, SEMA3C, HES5, and FGFRL1 compared to reference value ranges for the levels of the one or more biomarkers in a control aqueous humor sample indicate that the patient has a risk of age-related pathology and morbidity; and
increasing screening of the patient for an aging-related disease if the patient is identified as having the risk age-related pathology and morbidity.

115. The method of claim 114, further comprising administering a treatment for the aging-related disease to the patient if the patient is identified as having the aging-related disease.

116. A method of monitoring biological aging of an eye in a patient, the method comprising:
obtaining an aqueous humor sample from an eye of the patient; and
measuring levels of one or more biomarkers selected from stromelysin-2 (MMP10), neuropilin-1 (NRP1), semaphorin 3C (SEMA3C), hes family bHLH transcription factor 5 (HES5), and fibroblast growth factor receptor like 1 (FGFRL1) in the aqueous humor sample, wherein levels of the one or more biomarkers selected from MMP10, NRP1, SEMA3C, HES5, and FGFRL1 are correlated with biological age of the eye.

117. (canceled)

118. The method of claim 117, wherein the levels of MMP10, NRP1, SEMA3C, HES5, and FGFRL1 are measured in the aqueous humor sample.

119-128. (canceled)
